(19) United States
(12) Patent Application Publication
(10) Pub. No.: US 2005/0020605 A1
(43) Pub. Date: Jan. 27, 2005

(54) Quinolyl amide derivatives as CCR-5 antagonists

(75) Inventors: Shou-Fu Lu, San Ramon, CA (US); Gary Phillips, Pleasant Hill, CA (US); Bin Ye, Moraga, CA (US)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 10/865,976

(22) Filed: Jun. 10, 2004

Related U.S. Application Data
(60) Provisional application No. 60/477,940, filed on Jun. 13, 2003.

(51) Int. Cl. C 07D 4 1/04; C 07D 4 3/04; A61K 31/496; A61K 31/4545

(52) U.S. Cl. 514/253.01; 514/316; 544/360; 546/189

(57) ABSTRACT

The present invention relates to a series of compounds which are CCR-5 receptor antagonists of the general formula I: 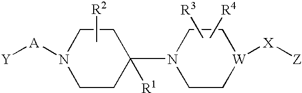 or a pharmaceutically acceptable salt thereof, wherein the variables are defined herein.

This application claims priority to U.S. Provisional Application Ser. No. 60/477,940 filed Jun. 13, 2003 the entirety of which is incorporated herein by reference.

### BACKGROUND OF THE INVENTION

Chemoattractant cytokines or chemokines are a family of proinflammatory mediators that promote recruitment and activation of leukocytes (e.g., monocytes, lymphocytes, and granulocytes). They can be released by many kinds of tissue cells after activation. Continuous release of chemokines at sites of inflammation mediates the ongoing migration of effector cells in chronic inflammation. The chemokines characterized to date are related in primary structure. They share four conserved cysteines, which form disulfide bonds. Based upon this conserved cysteine motif, the family is divided into two main branches, designated as the C--X--C chemokines (α-chemokines), and the C--C chemokines (β-chemokines), in which the first two conserved cysteines are separated by an intervening residue, or adjacent, respectively (Baggiolini, M. and Dahinden, C. A., Immunology Today, 15:127-133 (1994)).

The C--C chemokines include RANTES (Regulated on Activation, Normal T Expressed and Secreted), the macrophage inflammatory proteins 1α and 1β (MIP-1α and MIP-1β), and human monocyte chemotatic proteins 1-3 (MCP-1, MCP-2, MCP-3), which have been characterized as chemoattractants and activators of monocytes or lymphocytes. Chemokines, such as RANTES and MIP-1 a have been implicated in a wide range of human acute and chronic inflammatory diseases including rheumatoid arthritis, and respiratory diseases, such as asthma and allergic disorders. In particular a number of laboratories have implicated chemokines in the pathophysiology of RA (rheumatoid arthritis). Several studies involving human arthritic patients have demonstrated an increase in the expression levels of the CCR-5 ligands RANTES, MIP-1β, and MIP-1α in diseased synovium and an increased selective accumulation of CCR-5⁺ lymphocytes in diseased synovium fluid. (Rathanaswami P. et al., Journal of Biological Chemistry 268: 5834-9 (1993) and Rot A. et al. Journal of Experimental Medicine 176: 1489-95 (1992)).

The chemokine receptors are members of a superfamily of G protein-coupled receptors (GPCR) which share structural features that reflect a common mechanism of action of signal transduction (Gerard, C. and Gerard, N. P., Annu Rev. Immunol., 12:775-808 (1994); Gerard, C. and Gerard, N. P., Curr. Opin. Immunol., 6:140-145 (1994)). The first receptor for the C--C chemokines that was cloned and expressed binds the chemokines MIP-1α and RANTES. Accordingly, this MIP-1α/RANTES receptor was designated C--C chemokine receptor 1 (also referred to as CCR-1; Neote, K., et al., Cell, 72:415-425 (1993); Horuk, R. et al., WO 94/11504, May 26, 1994; Gao, J.-I. et al., J. Exp. Med., 177:1421-1427 (1993)). Three other receptors have been characterized which bind and/or signal in response to RANTES: CCR-3 mediates binding and signaling of chemokines including eotaxin, RANTES, and MCP-3 (Ponath et al., J. Exp. Med., 183:2437 (1996)), CCR-4 binds chemokines including RANTES, MIP-1α, and MCP-1 (Power, et al., J. Biol. Chem., 270:19495 (1995)), and CCR-5 binds chemokines including MIP-1α, RANTES, and MIP-1β. (Samson, et al., Biochem. 35: 3362-3367 (1996)).

RANTES is a chemotactic chemokine for a variety of cell types, including monocytes, eosinophils, and a subset of T-cells. The ability of RANTES to induce the directed migration of monocytes and a memory population of circulating T-cells (Schall, T. et al., Nature, 347:669-71 (1990)) suggests that this chemokine and its receptor(s) plays an important role in chronic inflammatory diseases, since these diseases are characterized by destructive infiltrates of T cells and monocytes.

### SUMMARY OF THE INVENTION

The present invention relates to a series of compounds which are CCR-5 receptor antagonists of the following formula I

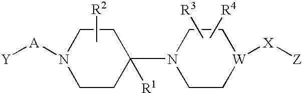

or a pharmaceutically acceptable salt thereof, wherein

Y is a 7 to 10 member bicyclic heterocycle optionally substituted with 1-3 independently selected moieties each of which is R⁵ or R⁶;

A is —CO—, or —SO₂—;

W is N or CH;

Z is R⁷-phenyl, R⁷-pyridyl, R⁷-thiophenyl or R⁷-naphthyl;

when W is CH then X is —C(R⁸)₂—, —C(R⁸)(R⁹)—, —C(O)—, —O—, —NH—, —N(C₁-₆ alkyl)-,

—C(R⁸)(OR¹⁰)—, —C(R⁸)(CH₂—C₁-₅alkyl-R¹⁰)—, —C(═CHR¹¹)—,

—C(═NOR¹²)—, —C(R⁸)(O—C₁-₆-alkyl)-, —C(═CH—C₁-₆ alkyl)-,

—C(R⁸)(O—C(O)—C₁-₆ alkyl)-, —C(R⁸)(O—C(O)—O—C₁-₆ alkyl)-,

—C(R⁸)(O—C(O)—NH—C₁-₆ alkyl)-, —C(R⁸)(O—C(O)—N(C₁-₆ alkyl)₂)-

—C(R⁸)(NR¹³—C(O)—C₁-₆ alkyl)-, —C(R⁸)(NR¹³—C(O)—O—C₁-₆ alkyl)-,

—C(R⁸)(NR¹³—C(O)—NH—C₁-₆ alkyl)-, —C(R⁸)(NR¹³—C(O)—N—(C₁-₆ alkyl)₂)-,

—N(C(O)—C₁-₆alkyl)-, —C(R⁸)(OH)—, —C(R⁸)(OTMS)-, —CHR⁸—, —CHR¹¹—, —CHR¹⁴—, — or

when W is N then X is —C(R⁸ )(R¹⁵)—, or —C(O)—;

R¹ is hydrogen, C₁-₆ alkyl or C₂-₆ alkenyl;

R², R³, R⁴, and R⁸ are each independently hydrogen, C₂-6 alkenyl, CF₃ or C₁-₆ alkyl;

R⁵ and R⁶ are independently selected from halogen, C₁-₆ alkyl, CF₃, nitro, cyano, NR¹³R¹¹, hydroxy, aryl, ester, carboxy, —CO₂R¹¹, OC₁-₆ alkyl;

R⁷ is 1 to 3 independently selected moieties each of which is hydrogen, halogen, nitro, —NR¹³R¹¹, —CF₃, CF₃O—, —CN, CF₃SO₂—, R¹⁹-phenyl, —NHCOCF₃, C₁-₆ alkyl, —CO₂C₁-₆ alkoxy, 5-membered heteroaryl, CH₃SO₂— or

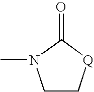

wherein Q is, —O—, —NH— or —N(CH₃)—;

R⁹ is R⁷-phenyl, R⁷-heteroaryl, R⁷-naphthyl, C₃-₁₀ cycloalkyl, C₃-₁₀ cycloalkyl —C₁-₆ alkyl or C₁-₆ alkoxy-C₁-₆ alkyl;

R¹⁰ is R¹⁷-phenyl, pyridyl, pyrimidyl, pyrazinyl or thiazolyl;

R¹¹ is H or C₁-₆ alkyl.

R¹² is hydrogen, —C₁-₆ alkyl, —C₁-₆ alkyl substituted by C₃-₇ cycloalkyl, —C₁-₆ alkyl, fluoro-C₁-₆ alkyl, cyclopropylmethyl-, —CH₂CH₂OH, —CH₂CH₂—O—C₁-₆ alkyl, —CH₂C(O)—O—C₁-₆ alkyl, —CH₂C(O)NH₂, —CH₂C(O)—NHC₁-₆ alkyl, —CH₂CH₂C₁-₆ alkyl, —CH₂C(O)—C₁-₆ alkyl or —CH₂C(O)—N(C₁-₆ alkyl)₂;

R¹³ is hydrogen or C₁-₆ alkyl;

R¹⁴ is —OH, —CF₃, or O-pyridinyl;

R¹⁵ is hydrogen, C₁-₆ alkyl, C₁-C₆ alkoxy-C₁-₆ alkyl, C₃-₁₀ cycloalkyl, C₃-₁₀ cycloalkyl-C₁-₆ alkyl, R¹⁶-phenyl, R¹⁶-phenyl-C₁-₆ alkyl, R¹⁶-naphthyl, R¹⁶-naphthyl-C₁-₆ alkyl, R¹⁶-heteroaryl or R¹⁶-heteroaryl-C₁-₆ alkyl;

R¹⁶ is 1 to 3 independently selected moieties each of which is hydrogen, halogen, C₁-₆ alkyl, C₁-₆-alkoxy, —CF₃, CF₃O—, CH₃C(O)—, —CN, CH₃SO₂—, CF₃SO₂—, R¹⁸-phenyl, R¹⁸-benzyl, CH₃C(═NOCH₃)—, CH₃C(═NOCH₂CH₃)—, —NH₂, —NHCOCF₃, —NHCONH—(C₁-₆ alkyl), —NHCO(C₁-₆ alkyl), —NHSO₂(C₁-₆ alkyl),5-membered heteroaryl, 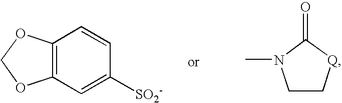
wherein Q is, —NH— or —N(CH₃);

R¹⁷ is C₁-₆ alkyl , —NH₂ or R¹⁹-phenyl-;

R¹⁸ is 1 to 3 independently selected moieties each of which is hydrogen, C₁-₆-alkyl, —CF₃, —CO₂H, —CO₂C₁-₆ alkoxy, —CN, C₁-₆ alkoxy or halogen;

R¹⁹ is 1 to 3 independently selected moieties each of which is hydrogen, C₁₋₆ alkyl, —CF₃, —CO₂R¹¹, —CN, C₁-₆ alkoxy or halogen;

The above formula includes separated chiral species, e.g., diastereomers and enantiomers, as well as all mixtures thereof, e.g., racemates, etc.

For the independently selected moieties mentioned above, all substituent patterns are envisioned.

The compounds of the present invention are useful in the prevention and treatment of a wide variety of inflammatory and immunoregulatory disorders and diseases, allergic conditions, atopic conditions, as well as autoimmune and immunodeficiency pathologies.

Also included in the invention are methods of using the compounds as agents for the treatment of CCR-5 mediated disease states, in particular for the treatment of inflammatory diseases or conditions, autoimmune disorders, and immune deficiency disorders such as HIV infection.

In another aspect, the instant invention may be used to evaluate specific antagonists of CCR-5 receptors. Accordingly, the present invention is directed to the use of these compounds in the preparation and execution of screening assays for compounds which modulate the activity of CCR-5 receptors. For example, the compounds of this invention are useful for isolating receptor mutants, which are excellent screening tools for more potent compounds. Furthermore, the compounds of this invention are useful in establishing or determining the binding site of other compounds to CCR-5 receptors, e.g., by competitive inhibition.

The compounds of the invention can be used in the treatment of mammals, preferably humans, comprising administering to such mammal in need thereof, an effective amount of a compound of formula (I), or a pharmaceutically acceptable salt thereof, optionally in the form of a separated diastereomer or enantiomer, e.g., less than 5%, 2%, or less of the other chiral entity(ies).

In preferred aspects, the invention relates to compounds, wherein Y is selected from the following groups, which in each case are optionally substituted:

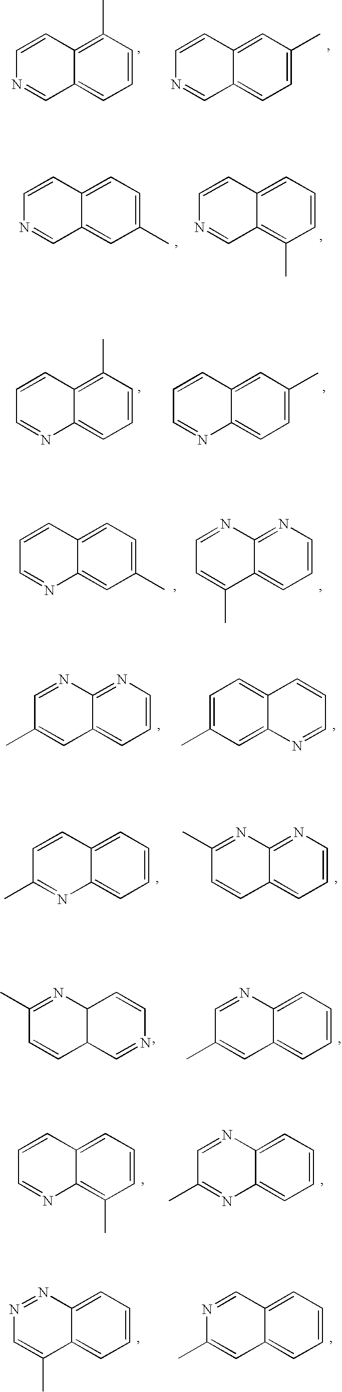

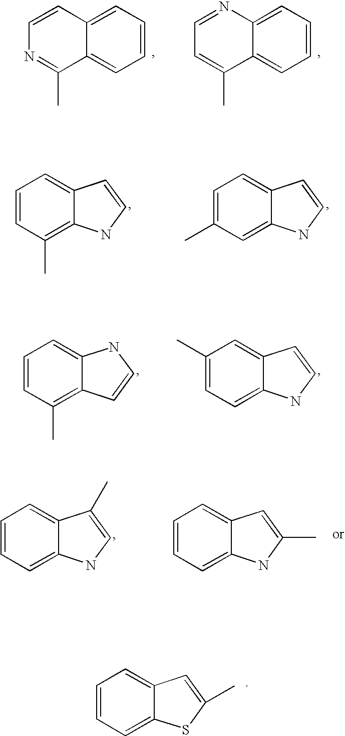

Also preferred are compounds of formula I, wherein Z is bromophenyl, trifluoromethylphenyl, or fluorophenyl.

Also preferred are compounds of formula I wherein X is
—C(═NHOEthyl)-
—CH(Opyridinyl)-
—CH(methyl)-
—C(═CH₂)— or
—CH(OH)—

Also preferred are compounds of formula I wherein R¹ is hydrogen or methyl.

Also preferred are compounds of formula I wherein Y may be substituted with one or more (e.g., 1-3) substituents which independently are chlorine, OH, C₁₋₆ alkyl , OMe, CF₃, phenyl or if Y is an N-heterocycle, the substituent may be an oxide of the nitrogen.

Other preferred embodiments of the present invention include:

a) A pharmaceutical composition comprising a compound of formula I in admixture with a pharmaceutically acceptable excipient, diluent, or carrier;

b) A method for modulation of chemokine receptor activity in a mammal which comprises administering an effective amount of a compound of formula I;

c) A method for the prevention or treatment of an inflammatory or immunoregulatory disorder or disease which comprises administering to a patient (e.g., mammal , e.g., human) an effective amount of a compound of formula I;

d) A method for the prevention or treatment of asthma, allergic rhinitis, dermatitis, conjunctivitis, or atherosclerosis which comprises administering to a patient an effective amount of a compound of formula I;

e) A method for the prevention or treatment of rheumatoid arthritis which comprises administering to a patient an effective amount of a compound of formula I;

f) A method for preventing infection by HIV, treating infection by HIV, delaying the onset of AIDS, or treating AIDS comprising administering to a patient an effective amount of a compound of formula I;

g) A method for the prevention or treatment of multiple sclerosis or psoriasis which comprises administering to a patient an effective amount of a compound of formula I;

h) A method of inhibiting the binding of MIP-1α or MIP-1β to a receptor comprising administering a therapeutically effective amount of a compound of formula I to a mammal in need thereof;

i) A method of inhibiting the binding of RANTES to a receptor comprising administering a therapeutically effective amount of a compound of formula I to a mammal in need thereof; and

j) A method of assaying compounds which modulate the activity of a CCR-5 receptor comprising screening against a compound of formula (1);

Preferred compounds of formula I are:

4-[[4-[4-(4-bromobenzoyl)-1-piperidinyl]-4-methyl-1-piperidinyl]carbonyl]-7-chloroquinoline;

1-hydroxy-4-[[4-methyl-4-[(3S)-3-methyl-4-[(1R)-1-[4-(trifluoromethyl)phenyl]ethyl]piperazinyl]-1-piperidinyl]carbonyl]quinolinium;

1-[[4-[4-[(4-bromophenyl)(ethoxyimino)methyl]-1-piperidinyl]-4 methyl-1-piperidinyl]carbonyl]isoquinoline;

3-[[4-[4-[(4-bromophenyl)(ethoxyimino)methyl]-1-piperidinyl]-4-methyl-1-piperidinyl]carbonyl]isoquinoline;

3-[[4-[4-[(4-bromophenyl)(ethoxyimino)methyl]-1-piperidinyl]-4-methyl-1-piperidinyl]carbonyl]quinoline;

2-[[4-[4-[(4-bromophenyl)(ethoxyimino)methyl]-1-piperidinyl]-4-methyl-1-piperidinyl]carbonyl]quinoline;

4-[[4-[4-[(4-bromophenyl)(ethoxyimino)methyl]-1-piperidinyl]-4-methyl-1-piperidinyl]carbonyl]-2-methyl-3-quinolinol;

4-[[4-[4-[(4-bromophenyl)(ethoxyimino)methyl]-1-piperidinyl]-4-methyl-1-piperidinyl]carbonyl]-8-methylquinoline;

4-[[4-[4-[(4-bromophenyl)(ethoxyimino)methyl]-1-piperidinyl]-4-methyl-1-piperidinyl]carbonyl]-6-methylquinoline;

2-[[4-[4-[(4-bromophenyl)(ethoxyimino)methyl]-1-piperidinyl]-4-methyl-1-piperidinyl]carbonyl]-4-quinolinol;

2-[[4-[4-[(4-bromophenyl)(ethoxyimino)methyl]-1-piperidinyl]-4-methyl-1-piperidinyl]carbonyl]-4,8-quinolinediol;

2-[[4-[4-[(4-bromophenyl)(ethoxyimino)methyl]-1-piperidinyl]-4-methyl-1-piperidinyl]carbonyl]-4-methoxyquinoline;

4-[[4-[4-[(E)-(4-bromophenyl)(ethoxyimino)methyl]-1-piperidinyl]-4-methyl-1-piperidinyl]carbonyl]-6methyl-5-quinolinol;

4-[[4-[4-[(E)-(4-bromophenyl)(ethoxyimino)methyl]-1-piperidinyl]-4-methyl-1-piperidinyl]carbonyl]-7-chloro-6-methylquinoline;

3-[[4-[4-[(4-bromophenyl)(ethoxyimino)methyl]-1-piperidinyl]-4-methyl-1-piperidinyl]carbonyl]-6-(trifluoromethyl)-7-quinolinol;

3-[[4-[4-[(Z)-(4-bromophenyl)(ethoxyimino)methyl]-1-piperidinyl]-4-methyl-1-piperidinyl]carbonyl]-6-(trifluoromethyl)-7-quinolinol;

3-[[4-[4-[(4-bromophenyl)(ethoxyimino)methyl]-1-piperidinyl]-4-methyl-1-piperidinyl]carbonyl]-8-(trifluoromethyl)-4-quinolinol;

2-[[4-[4-[(4-bromophenyl)(ethoxyimino)methyl]-1-piperidinyl]-4-methyl-1-piperidinyl]carbonyl]-6-ethyl-4-quinolinol;

3-[[4-[4-[(4-bromophenyl)(ethoxyimino)methyl]-1-piperidinyl]-4-methyl-1-piperidinyl]carbonyl]-7-(trifluoromethyl)-4-quinolinol;

2-[[4-[4-[(4-bromophenyl)(ethoxyimino)methyl]-1-piperidinyl]-4-methyl-1-piperidinyl]carbonyl]-8-methyl-4-quinolinol;

4-[[4-[4-[(4-bromophenyl)(ethoxyimino)methyl]-1-piperidinyl]-4-methyl-1-piperidinyl]carbonyl]-2-phenylquinoline;

6-[[4-[4-[(E)-(4-bromophenyl)(ethoxyimino)methyl]-1-piperidinyl]-4-methyl-1-piperidinyl]carbonyl]quinoline;

2-[[4-[4-[(4-bromophenyl)(ethoxyimino)methyl]-1-piperidinyl]-4-methyl-1-piperidinyl]carbonyl]-7-ethyl-4-quinolinol;

4-[[4-[4-[(Z)-(4-bromophenyl)(ethoxyimino)methyl]-1-piperidinyl]-4-methyl-1-piperidinyl]carbonyl]quinoline;

4-[[4-[4-[(4-bromophenyl)(ethoxyimino)methyl]-1-piperidinyl]-4-methyl-1-piperidinyl]carbonyl]-7-(trifluoromethyl)quinoline;

4-[[4-[4-[(Z)-(4-bromophenyl)(ethoxyimino)methyl]-1-piperidinyl]-4-methyl-1-piperidinyl]carbonyl]-1-hydroxyquinolinium;

7-[[4-[4-[(Z)-(4-bromophenyl)(ethoxyimino)methyl]-1-piperidinyl]-4-methyl-1-piperidinyl]carbonyl]quinoline;

8-[[4-[4-[(Z)-(4-bromophenyl)(ethoxyimino)methyl]-1-piperidinyl]-4-methyl-1-piperidinyl]carbonyl]quinoline;

4-[[4-[4-[(Z)-(4-bromophenyl)(ethoxyimino)methyl]-1-piperidinyl]-4-methyl-1-piperidinyl]carbonyl]-7-chloroquinoline;

4-[[4-[4-[(Z)-(4-bromophenyl)(ethoxyimino)methyl]-1-piperidinyl]-4-methyl-1-piperidinyl]carbonyl]-7-methylquinoline;

4-[[4-[4-[(E)-(4-bromophenyl)(ethoxyimino)methyl]-1-piperidinyl]-4-methyl-1-piperidinyl]carbonyl]-7-chloro-1-hydroxyquinolinium;

8-[[4-[4-[(Z)-(4-bromophenyl)(ethoxyimino)methyl]-1-piperidinyl]-4-methyl-1-piperidinyl]carbonyl]-4-chloroquinoline;

7-[[4-[4-[(Z)-(4-bromophenyl)(ethoxyimino)methyl]-1-piperidinyl]-4-methyl-1-piperidinyl]carbonyl]-4-chloroquinoline;

4-[[4-[4-[(Z)-(4-bromophenyl)(ethoxyimino)methyl]-1-piperidinyl]-4-methyl-1-piperidinyl]carbonyl]-2-methylquinoline;

5-[[4-[4-[(Z)-(4-bromophenyl)(ethoxyimino)methyl]-1-piperidinyl]-4-methyl-1-piperidinyl]carbonyl]-1-hydroxyquinolinium;

4-[[4-[4-[(E)-(4-bromophenyl)(ethoxyimino)methyl]-1-piperidinyl]-4-methyl-1-piperidinyl]carbonyl]-7-methoxyquinoline;

5-[[4-[4-[(E)-(4-bromophenyl)(ethoxyimino)methyl]-1-piperidinyl]-4-methyl-1-piperidinyl]carbonyl]quinoline;

2-[[4-[[4-[4-[(Z)-(4-bromophenyl)(ethoxyimino)methyl]-1-piperidinyl]-4-methyl-1-piperidinyl]carbonyl]-7-quinolinyl]oxy]ethanol

4-[[4-[4-[(Z)-(4-bromophenyl)(ethoxyimino)methyl]-1-piperidinyl]-4-methyl-1-piperidinyl]carbonyl]-3-methylquinoline;

8-[[4-[4-[(Z)-(4-bromophenyl)(ethoxyimino)methyl]-1-piperidinyl]-4-methyl-1-piperidinyl]sulfonyl]quinoline;

4-[[4-[4-[(4-bromophenyl)(ethoxyimino)methyl]-1-piperidinyl]-4-methyl-1-piperidinyl]carbonyl]quinoline;

2-[[4-[4-[(4-bromophenyl)(ethoxyimino)methyl]-1-piperidinyl]-4-methyl-1-piperidinyl]carbonyl]quinoxaline;

2-[[4-[4-[(4-bromophenyl)(ethoxyimino)methyl]-1-piperidinyl]-4-methyl-1-piperidinyl]carbonyl]-3-quinoxalinol;

2-[[4-[4-[(4-bromophenyl)(ethoxyimino)methyl]-1-piperidinyl]-4-methyl-1-piperidinyl]carbonyl]-1,6-naphthyridine;

2-[[4-[4-[(4-bromophenyl)(ethoxyimino)methyl]-1-piperidinyl]-4-methyl-1-piperidinyl]carbonyl]-1,8-naphthyridine;

3-[[4-[4-[(E)-(4-bromophenyl)(ethoxyimino)methyl]-1-piperidinyl]-4-methyl-1-piperidinyl]carbonyl]-2-methyl-1,8-naphthyridine;

3-[[4-[4-[(4-bromophenyl)(ethoxyimino)methyl]-1-piperidinyl]-4-methyl-1-piperidinyl]carbonyl]-2-(trifluoromethyl)-1,8-naphthyridine;

3-[[4-[4-[(Z)-(4-bromophenyl)(ethoxyimino)methyl]-1-piperidinyl]-4-methyl-1-piperidinyl]carbonyl]-2-methyl-1,6-naphthyridine;

2-[[4-[4-[(4-bromophenyl)(ethoxyimino)methyl]-1-piperidinyl]-4-methyl-1-piperidinyl]carbonyl]-1H-indole;

3-[[4-[4-[(4-bromophenyl)(ethoxyimino)methyl]-1-piperidinyl]-4-methyl-1-piperidinyl]carbonyl]-1-methyl-1H-indole;

3-[[4-[4-[(4-bromophenyl)(ethoxyimino)methyl]-1-piperidinyl]-4-methyl-1-piperidinyl]carbonyl]-1H-indole;

5-[[4-[4-[(4-bromophenyl)(ethoxyimino)methyl]-1-piperidinyl]-4-methyl-1-piperidinyl]carbonyl]-1H-indole;

5-[[4-[4-[(Z)-(4-bromophenyl)(ethoxyimino)methyl]-1-piperidinyl]-4-methyl-1-piperidinyl]carbonyl]-1-methyl-1H-indole;

5-[[4-[4-[(Z)-(4-bromophenyl)(ethoxyimino)methyl]-1-piperidinyl]-4-methyl-1-piperidinyl]carbonyl]-1-ethyl-1H-indole;

2-[[4-[4-[(E)-(4-bromophenyl)(ethoxyimino)methyl]-1-piperidinyl]-4-methyl-1-piperidinyl]carbonyl]-1-methyl-1H-indole;

2-[[4-[4-[(4-bromophenyl)(ethoxyimino)methyl]-1-piperidinyl]-4-methyl-1-piperidinyl]carbonyl]-1-ethyl-1H-indole;

3-[[4-[4-[(Z)-(4-bromophenyl)(ethoxyimino)methyl]-1-piperidinyl]-4-methyl-1-piperidinyl]carbonyl]-1-ethyl-1H-indole;

4-[[4-[4-[(Z)-(4-bromophenyl)(ethoxyimino)methyl]-1-piperidinyl]-4-methyl-1-piperidinyl]carbonyl]-1-methyl-1H-indole;

6-[[4-[4-[(Z)-(4-bromophenyl)(ethoxyimino)methyl]-1-piperidinyl]-4-methyl-1-piperidinyl]carbonyl]-1-methyl-1H-indole;

4-[[4-[4-[(Z)-(4-bromophenyl)(ethoxyimino)methyl]-1-piperidinyl]-4-methyl-1-piperidinyl]carbonyl]-1-ethyl-1H-indole;

6-[[4-[4-[(Z)-(4-bromophenyl)(ethoxyimino)methyl]-1-piperidinyl]-4-methyl-1-piperidinyl]carbonyl]-1-ethyl-1H-indole;

6-[[4-[4-[(Z)-(4-bromophenyl)(ethoxyimino)methyl]-1-piperidinyl]-4-methyl-1-piperidinyl]carbonyl]-1H-indole;

4-[[4-[4-[(Z)-(4-bromophenyl)(ethoxyimino)methyl]-1-piperidinyl]-4-methyl-1-piperidinyl]carbonyl]-1-methyl-1H-indole;

1-[1-(benzo[b]thien-3-ylcarbonyl)-4-methyl-4-piperidinyl]-4-[(4-bromophenyl)(ethoxyimino)methyl]piperidine;

4-[[4-[4-[(4-bromophenyl)hydroxy-methyl]-4-(4-methyl-4-piperidinyl)-piperidinyl-quinoline;

4-[[4-[4-[(4-bromophenyl)(2-pyridinyloxy)methyl]-1-piperidinyl]-4-methyl-1-piperidinyl]carbonyl]-7-chloroquinoline;

4-[[4-[4-[(4-bromophenyl)(2-pyridinyloxy)methyl]-1-piperidinyl]-4-methyl-1-piperidinyl]carbonyl]quinoline;

4-[[4-[4-[(4-bromophenyl)(2-pyridinyloxy)methyl]-1-piperidinyl]-4-methyl-1-piperidinyl]carbonyl]-1-hydroxyquinolinium;

5-[[4-[4-[(4-bromophenyl)(2-pyridinyloxy)methyl]-1-piperidinyl]-4-methyl-1-piperidinyl]carbonyl]quinoline;

5-[[4-[4-[(4-bromophenyl)(2-pyridinyloxy)methyl]-1-piperidinyl]-4-methyl-1-piperidinyl]carbonyl]-1-hydroxyquinolinium;

4-[[4-[4-[1-(4-bromophenyl)-2,2,2-trifluoro-1-[(trimethylsilyl)oxy]ethyl]-1-piperidinyl]-4-methyl-1-piperidinyl]carbonyl]-7-chloroquinoline;

4-[[4-[4-[1-(4-bromophenyl)-(2,2,2-trifluoro-1-hydroxy)ethyl]-1-piperidinyl]-4-methyl-1-piperidinyl]carbonyl]-7-chloroquinoline

4-[[4-[4-[1-(4-bromophenyl)ethenyl]-1-piperidinyl]-4-methyl-1-piperidinyl]carbonyl]quinoline;

1-methyl-4-[[4-methyl-4-[(3S)-3-methyl-4-[(1R)-1-[4-(trifluoromethyl)phenyl]ethyl]piperazinyl]-1-piperidinyl]carbonyl]-1H-indole;

or pharmaceutically acceptable salts thereof, wherein these compounds can be in the form of individual optical isomers or mixtures thereof such as diastereomeric mixtures or racemic mixtures.

Other preferred embodiments of the present invention include:

The term “bicyclic heteroaryl” in Y include stable 7- to 10-membered fused bicyclic rings which may be either saturated or unsaturated, and may comprise from 1 to 3 N, O and/or S, heteroatoms. Examples of such bicyclic heteroaryls include, but are not limited to, bicyclic rings such as naphthyridine, benzofuran, benzothiophene, indole, 1H-indazole, indoline, benzopyrazole, purine, quinoline, isoquinoline, benzimidazole, quinazoline, pyrido[2,3b]-pyrazine, pyrido[3,4]pyrazine, pyrido[3,2c]pyridazine, pyrido[3,4-b]-pyridine, pteridine, quinolone, isoquinolone, benzothiazole, quinoxaline, quinoline-N-oxide, isoquinoline-N-oxide, quinoxaline-N-oxide, quinazoline-N-oxide, benzoxazine, phthalazine, and cinnoline. The bicyclic heterocycle rings described herein may be substituted on a carbon or nitrogen atom if the resulting compound is stable. The nitrogen and sulfur heteroatoms may optionally be oxidized. Suitable substituents for the nitrogen heteroatom(s) include C₁-C₆ alkyl. The bicyclic heteroaryl ring can also be additionally substituted at any available carbon atom by C₁-C₆ alkyl, halogen, hydroxy, phenyl, aryl, ester (e.g., alkyl ester), alkoxy, CF₃, cyano, carboxy and/or nitro. It will be understood that the Y group substituent(s) may be the same or different and may be at any open position on its rings.

The term “alkyl” is used herein at all occurrences (or a group per se or a part of a group) to mean straight or branched chain alkyl groups of 1 to 6 carbon atoms, unless the chain length is otherwise limited, including, but not limited to methyl, ethyl, n-propyl, isopropyl, n-butyl, sec-butyl, isobutyl, tert-butyl, and the like. Alkyl groups may also be substituted one or more times by halogen, aryl, substituted aryl, hydroxy, methoxy, amino, substituted amino, nitro, carboxy, or cyano.

Alkoxy groups means alkyl-O— groups in which the alkyl portion (substituted or unsubstituted) is in accordance with the previous discussion. Suitable alkoxy groups are methoxy, ethoxy, propoxy and butoxy.

TMS means trimethylsilyl.

Alkenyl represents C₂-C₆ carbon chains having one or two unsaturated bonds, provided that two unsaturated bonds are not adjacent to each other.

Heteroaryl represents monocyclic aromatic groups of 5 or 6 atoms or bicyclic aromatic groups of 8 to 12 atoms having 1 to 3 O, S or N heteroatoms, said heteroatom(s) interrupting a carbocyclic ring structure and having a sufficient number of delocalized pi electrons to provide aromatic character, provided that the rings do not contain adjacent oxygen and/or sulfur atoms. Nitrogen atoms can be in the form of an N-oxide. All regioisomers are contemplated. Suitable 6-membered heteroaryl groups are pyridyl, pyrimidinyl, pyrazinyl, pyridazinyl and the N-oxides thereof. Suitable 5-membered heteroaryl rings are furyl, thienyl, pyrrolyl, thiazolyl, isothiazolyl, imidazolyl, pyrazolyl and isoxazolyl. 5-Membered rings having one heteroatom can be joined through the 2- or 3-position; 5-membered rings having two heteroatoms are preferably joined through the 4-position, in all cases using IUPAC nomemclature. Bicyclic groups typically are benzo-fused ring systems derived from the heteroaryl groups named above, e.g. quinolyl, phthalazinyl, quinazolinyl, benzofuranyl, benzothienyl and indolyl.

Suitable substituents on the amino groups herein can be the same or different and include alkyl (optionally substituted), and cycloalkyl (optionally substituted). Typical substituents include OH and C₁₋₆ alkoxy.

The term “cycloalkyl” is used herein at all occurrences to mean cyclic aliphatic radicals, preferably of 3 to 8 carbons, including but not limited to cyclopropyl, cyclopentyl, cyclohexyl, cycloheptyl and the like. These groups can also contain one to three (as appropriate) double bonds to form the “cycloalkenyl” groups e.g., cyclohexenel. Suitable substituents are halogen, C₁₋₆ alkyl, substituted alkyl, aryl, substituted aryl, arylalkyl, substituted arylalkyl, alkylcarbonyl, hydroxy, alkoxy, amino, substituted amino, nitro, carboxy, or cyano.

The terms “halo” or “halogen” are used interchangeably herein at all occurrences to mean radicals derived from the elements chlorine, fluorine, iodine or bromine. “Halogenated” is analogous and refers to a degree of halogen substitutions from single to full (per) substitution. Fluoro-(C₁-C₆)-alkyl represents a straight or branched alkyl chain substituted by 1 to 5 fluoro atoms, which can be attached to the same or different carbon atoms, e.g., —CH₂F, —CHF₂, —CF₃, F₃CCH₂— and —CF₂CF₃.

The terms “aryl” is used herein at all occurrences to mean 5-10 membered (fused or connected) aromatic ring(s) or ring systems which may include bi- or tri-cyclic systems. Aryl may also include heteroaryl as defined herein. Representative examples include, but are not limited to, phenyl, and naphthyl. Substituted aryl groups may be substituted one or more times by halogen, C₁₋₆ alkyl, hydroxy, alkoxy, e.g., methoxy, amino, substituted amino, nitro, methylene, trifluoromethyl, oxo, carboxy, or cyano.

Aryl alkyl is a aryl-alkyl radical wherein the aryl and alkyl portions are in accordance with the descriptions above.

It will be understood throughout that the optional substituents are selected independently from one another.

Some of the compounds of Formula I and related compounds are capable of forming both pharmaceutically acceptable acid addition and/or base salts. All of these forms are within the scope of the present invention, as are separated diastereomers and enantiomers.

The optical isomers can be obtained by resolution of the racemic mixtures according to conventional processes, for example, by the formation of diastereoisomeric salts using an optically active acid or base, or by formation of covalent diastereomers. Examples of appropriate optically active acids are tartaric, diacetyltartaric, dibenzoyltartaric, ditoyluoytartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids may then be liberated from the separated diastereomeric salts. A different process for separation of optical isomers involves use of chiral chromatography (e.g., chiral HPLC columns), with or without conventional derivitization, optimally chosen to maximize the separation of the enantiomers. Suitable chiral HPLC columns are manufactured by Diacel, e.g., Chiracel OD and Chiracel OJ, among many others, all routinely selectable. Enzymatic separations, with or without derivitization, are also useful. The optically active compounds of formula I can likewise be obtained by utilizing optically active starting materials.

Pharmaceutically acceptable acid addition salts of the compounds of Formula I include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, 2-phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al., “Pharmaceutical Salts,” J. Pharma. Sci., 1977;66:1).

The acid addition salts of basic compounds of formula I can be prepared by contacting the free base form with a sufficient amount of the desired acid to produce the salt in the conventional manner. The free base form may be regenerated by contacting the salt form with a base and isolating the free base in the conventional manner. The free base forms can differ from their respective salt forms somewhat in certain physical properties such as solubility in polar solvents.

Pharmaceutically acceptable base addition salts of the compounds of formula I can be formed with metals or amines, such as alkali and alkaline earth metals or organic amines. Examples of such metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see Berge, Supra, 1977).

The base addition salts of acidic compounds of formula I can be prepared by contacting the free acid form with a sufficient amount of the desired base to produce the salt in the conventional manner. The free acid form may be regenerated by contacting the salt form with an acid and isolating the free acid in the conventional manner. The free acid forms can differ from their respective salt forms somewhat in certain physical properties such as solubility in polar solvents.

Certain of the compounds of the present invention can exist in unsolvated forms as well as solvated forms, including hydrated forms. Solvated and unsolvated forms are intended to be encompassed within the scope of the present invention.

Certain of the compounds of the present invention possess one or more chiral centers and each center may exist in the R(D) or S(L) configuration. The present invention includes all diastereomeric, enantiomeric and epimeric forms as well as all mixtures thereof such as racemic mixtures.

The activity of compounds of the present invention can be assessed using suitable assays, such as receptor binding assays and chemotaxis assays. For example, as described in the example section, antagonist compounds of the present invention have been identified utilizing a CCR-5 Receptor MIP1α SPA binding assay and have been found to exhibit IC₅₀ values ranging from 0.05 μM to 38 μM. Such values are indicative of the intrinsic activity of the compounds in use as modulators of chemokine receptor activity. There are numerous other such screening assays known to those skilled in the art which may be used to determine the CCR-5 receptor antagonistic activity of the compounds of the present invention. One such screening technique is described in PCT WO 92/01810. Another assay, for example, may be employed for screening a receptor antagonist by contacting melanophore cells which encode the CCR-5 receptor with both the RANTES and a compound to be screened. Inhibition of the signal generated by the ligand indicates that a compound is an antagonist for the receptor, i.e., inhibits activation of the receptor.

Other screening techniques include the use of cells which express the CCR-5 receptor (for example, transfected CHO cells, RBL-2 cells or other mammalian cells) in a system which measures extracellular pH changes caused by receptor activation, for example, as described in Science, volume 246, pages 181-296 (October 1989), herein incorporated by reference. Potential antagonists may be contacted with a cell which expresses the CCR-5 receptor and a second messenger response, e.g. signal transduction or pH changes, or making use of a reporter gene system, for example luciferase, may be measured to determine whether the potential antagonist is effective.

Another such screening technique involves introducing mRNA encoding the CCR-5 receptor into Xenopus oocytes, RBL-2 or other mammalian cells to transiently express the receptor. The cells with the expressed receptor may then be contacted in the case of antagonist screening with RANTES and a compound to be screened, followed by detection of inhibition of a calcium or cAMP signal.

Another screening technique involves expressing the CCR-5 receptor in which the receptor is linked to a phospholipase C or D. As representative examples of such cells, there may be mentioned endothelial cells, smooth muscle cells, embryonic kidney cells, etc. The screening for an antagonist may be accomplished as herein above described by detecting inhibition of activation of the receptor from the phospholipase second signal.

Another method involves screening for CCR-5 receptor inhibitors by determining inhibition of binding of labeled RANTES to cells or membranes which have the receptor on the surface thereof. Such a method involves transfecting a eukaryotic cell, such as CHO or RBL-2 cell, with DNA encoding the CCR-5 receptor such that the cell expresses the receptor on its surface and contacting the cell with a potential antagonist in the presence of a labeled form of RANTES. The RANTES can be labeled, e.g., by radioactivity. The amount of labeled ligand bound to the receptors is measured, e.g., by measuring radioactivity associated with transfected cells or membrane from these cells. If the potential antagonist binds to the receptor, as determined by a reduction of labeled ligand which binds to the receptors, the binding of labeled ligand to the receptor is inhibited.

Another method involves screening for CCR-5 inhibitors by determining inhibition or stimulation of CCR-5-mediated cAMP and/or adenylate cyclase accumulation or diminution. Such a method involves transfecting a eukaryotic cell, such as CHO or RBL-2 cell, with CCR-5 receptor to express the receptor on the cell surface. The cell is then exposed to potential antagonists in the presence of RANTES. The amount of cAMP accumulation is then measured. If the potential antagonist binds the receptor, and thus inhibits CCR-5 binding, the levels of CCR-5-mediated cAMP, or adenylate cyclase, activity will be reduced or increased.

Another such screening technique is described in U.S. Pat. No. 5,928,881, which provides a method for determining whether a ligand not known to be capable of binding to the CCR-5 receptor can bind to such receptor which comprises contacting a mammalian cell which expresses the CCR-5 receptor with RANTES under conditions permitting binding of ligands to the CCR-5 receptor, detecting the presence of a ligand which binds to the receptor and thereby determining whether the ligand binds to the CCR-5 receptor.

A review of the role of chemokines in allergic inflammation is provided by Kita, H., et al., J. Exp. Med. 183, 2421-2426 (1996) suggesting that agents which modulate chemokine receptors would be useful in allergic inflammatory disorders and diseases. Compounds which modulate chemokine receptors are especially useful in the treatment and prevention of atopic conditions including allergic rhinitis, dermatitis, conjunctivitis, and particularly bronchial asthma.

Migration of leukocytes from blood vessels into diseased tissues is important to the initiation of normal disease-fighting inflammatory responses. But this process, known as leukocyte recruitment, is also involved in the onset and progression of debilitating and life-threatening chronic inflammatory, allergic inflammatory and autoimmune diseases. Thus, compounds which block leukocyte recruitment to target tissues in inflammatory and autoimmune disease would be a highly effective therapeutic intervention.

It has recently been recognized that for efficient entry into target cells, human immunodeficiency viruses require chemokine receptors, most probably CCR-5 or CXCR4, as well as the primary receptor CD4 (Levy, N. Engl. J. Med., 335(20), 1528-1530 (Nov. 14, 1996). The principal cofactor for entry mediated by the envelope glycoproteins of certain strains of HIV-1 is CCR-5, a receptor for the chemokines RANTES, MIP-1α and MIP-10 (Deng, et al., Nature, 381, 661666 (1996)). Accordingly, an agent which could block chemokine receptors in humans who possess normal chemokine receptors will prevent infection in healthy individuals and slow or halt viral progression in infected patients. Inhibition of chemokine receptors presents a viable method for the prevention or treatment of infection by HIV and the prevention or treatment of AIDS.

Small molecule antagonists of the interaction between C--C chemokine receptors and their ligands, including RANTES and MIP-1α, provide compounds useful for blocking chemokine receptors and inhibiting harmful inflammatory processes “triggered” by receptor ligand interaction, as well as valuable tools for the investigation of receptor-ligand interactions.

The selective inhibition of a CCR-5 receptor by treatment with the receptor antagonists of the invention represents a novel therapeutic and/or preventative approach to the treatment of a broad spectrum of inflammatory and autoimmune diseases or conditions, in particular for the treatment of inflammatory diseases or conditions, atherosclerosis, restenosis, and autoimmune disorders such as arthritis and transplant rejection.

In a preferred embodiment, the disease or condition is one which is associated with lymphocyte and/or monocyte infiltration of tissues (including recruitment and/or accumulation in tissues), such as arthritis (e.g., rheumatoid arthritis), inflammatory bowel diseases (e.g., Crohn's disease, ulcerative colitis), multiple sclerosis, idiopathic pulmonary fibrosis, and graft rejection (e.g., in transplantation), including allograft rejection or graft-versus-host disease. In addition, diseases characterized by basophil activation and/or eosinophil recruitment, including allergic hypersensitivity disorders such as psoriasis, asthma and allergic rhinitis can be treated according to the present invention.

Other diseases that may be treated with the compounds of Formula I are: chronic contact dermatitis, sarcoidosis, dermatomyositis, skin phemphigoid and related diseases (e.g., pemphigus vulgaris, p. foliacious, p. erythematosus), glomerulonephritides, vasculitides (e.g., necrotizing, cutaneous, and hypersensitivity vasculitis), hepatitis, diabetes, systemic lupus erythematosus and myasthenia gravis.

In addition to psoriasis, other inflammatory dermatoses such as dermatitis, eczema, atopic dermatitis, allergic contact dermatitis, urticaria and reperfusion injury can also be treated.

The antagonists of the present invention bind to the CCR-5 receptor, making it inaccessible to ligands such that normal biological activity is prevented. They may be administered to a mammal in need of treatment of CCR-5 mediated disease states. Thus, the active ingredient may be administered in the mammal using conventional course of treatment determination tests.

The term “CCR-5 mediated disease state” is used herein at all occurrences to mean any disease state which is affected or modulated by CCR-5.

The subject treated in the methods above is preferably a mammal, preferably a human being, male or female, in whom modulation of chemokine receptor activity is desired. “Modulation” as used herein is intended to encompass antagonism, agonism, partial antagonism, inverse agonism and/or partial agonism. In a preferred aspect of the present invention, modulation refers to antagonism of chemokine receptor activity, since the compounds of the invention are antagonists.

Combined therapy to modulate chemokine receptor activity and thereby prevent and treat the above-noted conditions illustrated by the combination of the compounds of this invention and other compounds which are known for such utilities. For example, in the treatment or prevention of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, and/or a cytokine-suppressing antiinflammatory agent, for example with a compound such as acetaminophen, aspirin, codeine, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, epinephrine, naphazoline, xylometazoline, propylhexedrine, or levo-desoxy-ephedrine; an antitussive such as codeine, hydrocodone, caramiphen, carbetapentane, or dextromethorphan; a diuretic; and/or a sedating or non-sedating antihistamine. Likewise, compounds of the present invention may be used in combination with other drugs that are used in the treatment/prevention/suppression or amelioration of the diseases or conditions for which compounds of the present invention are also useful. Such other drugs may be administered, by a route and in an amount commonly used therefor, together, contemporaneously or sequentially with a compound of the present invention. When a compound of the present invention is administered together with one or more other drug, they may be given sequentially or simultaneoulsy. When a compound of the present invention is used contemporaneously with one or more other drugs, a pharmaceutical composition containing such other drugs in addition to the compound of the present invention is preferred.

Accordingly, the pharmaceutical compositions of the present invention include those that also contain one or more other active ingredients, in addition to a compound of the present invention. Examples of other active ingredients that may be combined with a compound of the present invention, either administered separately or in the same pharmaceutical compositions, include, but are not limited to: (a) VLA-4 antagonists such as those described in U.S. Pat. No. 5,510,332, (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as .beta.2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, caiprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CXCRA, CCR-1, CCR-2, CCR-3 and CCR-5; (j) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (metformin), α-glucosidase inhibitors (acarbose) and glitazones (troglitazone and pioglitazone); (I) preparations of interferon beta (interferon-beta-lac, interferon-beta-1.beta.); (m) other compounds such as 5-aminosalicylic acid and prodrugs thereof, antimetabolites such as azathioprine and 6-mercaptopurine, and cytotoxic cancer chemotherapeutic agents.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient is preferably used.

The compounds of the present invention may be administered by oral, parenteral (e.g., intramuscular, intraperitoneal, intravenous, ICV, intracisternal injection or infusion, subcutaneous injection, or implant), inhalation (e.g., spray), nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. The compounds of the invention are effective for use in primates, such as humans, as well as for the treatment of warm-blooded animals such as mice, rats, horses, cattle, sheep, dogs, cats, monkeys, guinea pigs, other bovine, ovine, equine, canine, feline, rodent or murine species. However, the compounds of the invention are also effective for use in other species, such as avian species (e.g., chickens).

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.) The pharmaceutical composition and method of the present invention may further comprise other therapeutically active compounds as noted herein which are usually applied in the treatment of the above mentioned pathological conditions.

The compounds of the invention, or their pharmaceutically acceptable salts, are administered in a therapeutically effective amount which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disease-states; and the host undergoing therapy. Generally, a therapeutically effective daily dose is from about 0.14 mg to about 14.3 mg/kg of body weight per day of a compound of the invention, or a pharmaceutically acceptable salt thereof; preferably, from about 0.7 mg to about 10 mg/kg of body weight per day; and most preferably, from about 1.4 mg to about 7.2 mg/kg of body weight per day. For example, for administration to a 70 kg person, the dosage range would be from about 10 mg to about 1.0 gram per day of a compound of the invention, or a pharmaceutically acceptable salt thereof, preferably from about 50 mg to about 700 mg per day, and most preferably from about 100 mg to about 500 mg per day. The compounds may be administered on a regimen of 1 to 4 times per day, preferably once or twice per day.

In the foregoing and in the following examples, all temperatures are set forth uncorrected in degrees Celsius; and, unless otherwise indicated, all parts and percentages are by weight.

Compounds of the invention can be made by procedures known in the art, such as those disclosed in WO 00/66559; WO00/66558; WO 02/079157, and WO 02/079194. With respect to identified subgenuses and procedures of making, applicants incorporate by reference the entire disclosures of WO 00/66559; WO00/66558; WO 02/079157; and WO 02/079194, as if fully set forth herein. Furthermore, the entire disclosures of all applications, patents and publications, cited above or below, are hereby incorporated by reference.

Compounds of the invention can also be prepared as described in the following reaction schemes and by the methods described in the examples below.

General Methods of Preparation

Specifically, the compounds of the invention are prepared according to the following general methods and schemes:

1. General Method for the Preparation of Substituted-Quinolyl Acid

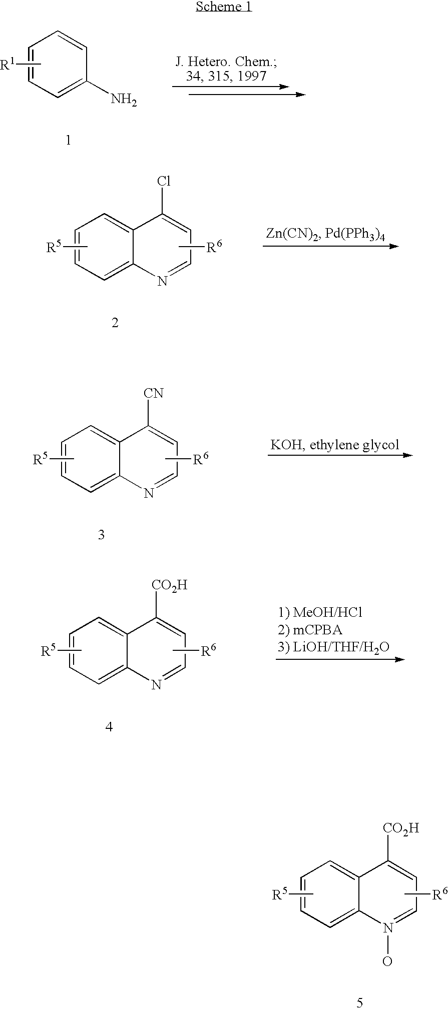

In Scheme 1, substituted 4-chloroquinoline 2 was synthesized from aniline 1 according to the known procedure (J. Heterocyclic Chemistry, 34, 315-320, 1997). Replacement of chloride by cyano with Zn(CN)₂ was achieved in under the catalysis of Pd(PPh₃)₄. Hydrolysis of 4-cyanoquinoline 3 using KOH in ethylene glycol afforded 4-quinolyl acid 4. Conversion of 4-quinolyl-acid 4 to N-oxide 5 was achieved through a three-step reaction: a) methyl ester formation in methanol solution with HCl; b) oxidation with mCPBA; c) hydrolysis of N-oxide methyl ester.

2. General Method for the Preparation of Oxime-Containing Amides

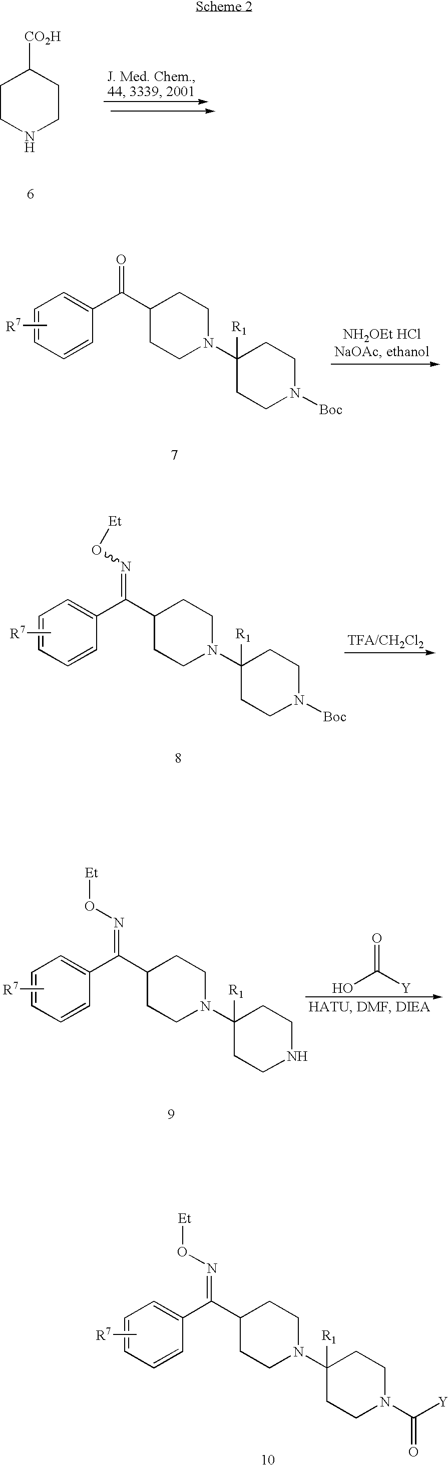

In Scheme 2, intermediate 7 was synthesized from isonipecotic acid 6 according to the known procedure (J. Med. Chem., 44, 3339-3342, 2001). Reaction of 7 with NH₂OEt.HCl in refluxing ethanol afforded oxime 8. Z and E isomers can be separated by column chromatography. Deprotection of 8 with TFA gave amine 9, which was converted to the final product 10 using HATU as an activator for the coupling with acids.

3. General Method for the Preparation of Pyridyl-Containing Amides

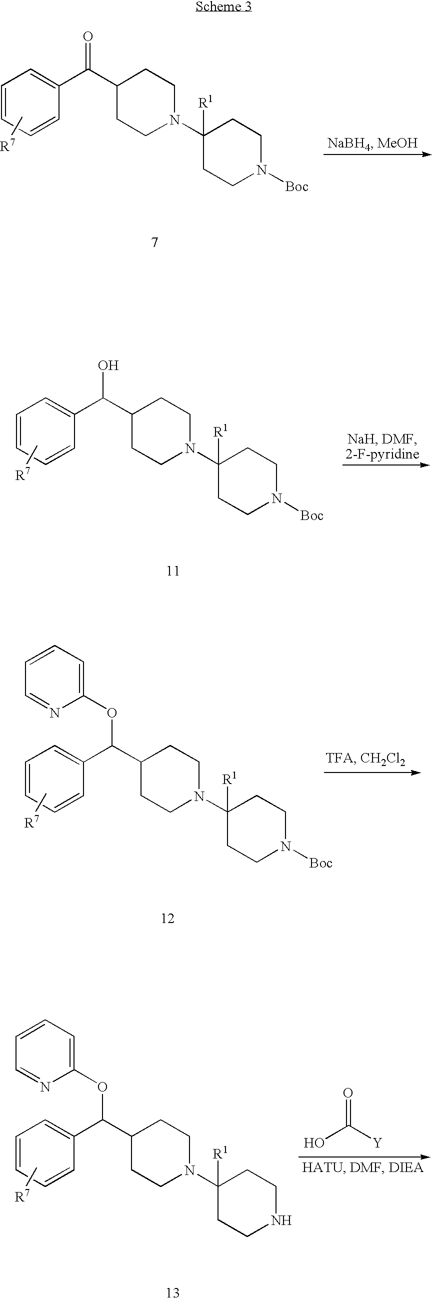

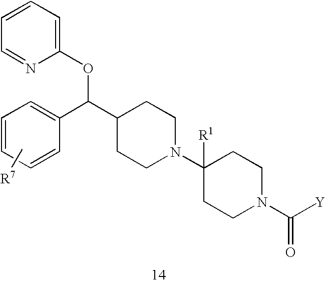

In Scheme 3, reduction of 7 with NaBH₄ in methanol afforded 11, which reacted further with 2-fluoropyridine using NaH as base to afford 12. Deprotection of 12 with TFA afforded free amine 13. Coupling of 13 with acids using HATU as an activator afforded final product 14

4. General Method for the Introduction of Trifluoromethyl Group

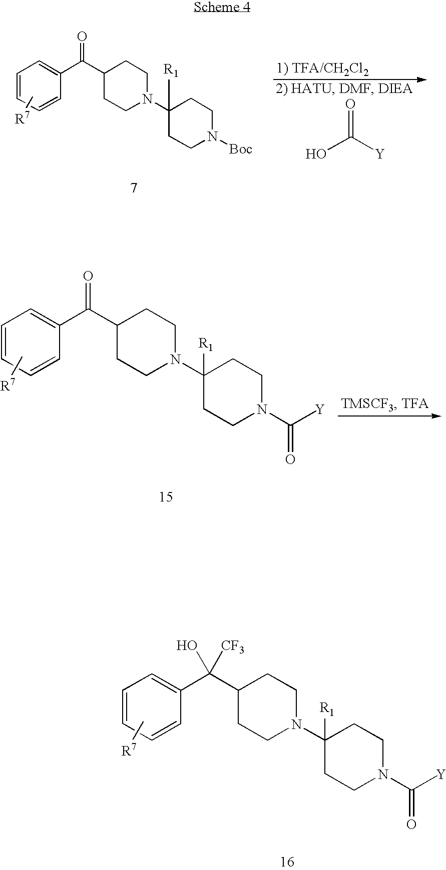

In Scheme 4, deprotection of 7 with TFA, followed by the coupling with acids using HATU as an activator afforded 15. Reaction of 15 with TMSCF₃ in the presence of TFA afforded 16.

5. General Method for the Introduction of Olefin Group

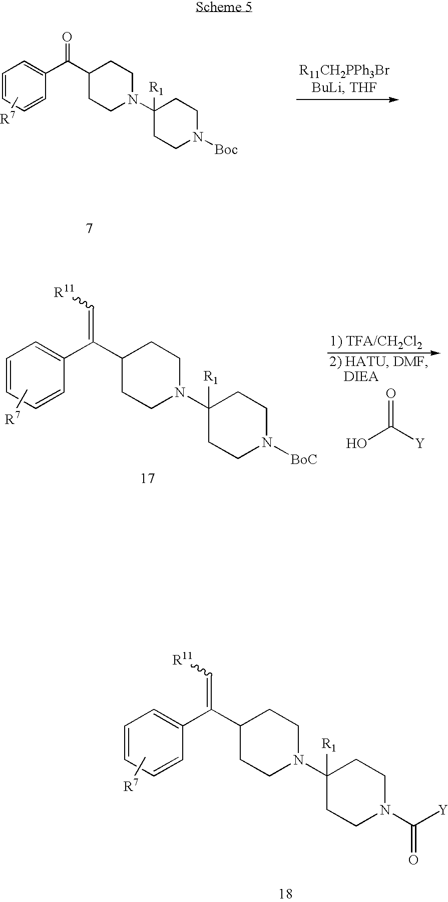

In Scheme 5, reaction of 7 with Wittig reagent afforded 17. Deprotection of 17 with TFA, followed by coupling with acids using HATU as an activator afforded 18.

6. General Method for the Preparation of Piperazine-Piperidine Amides

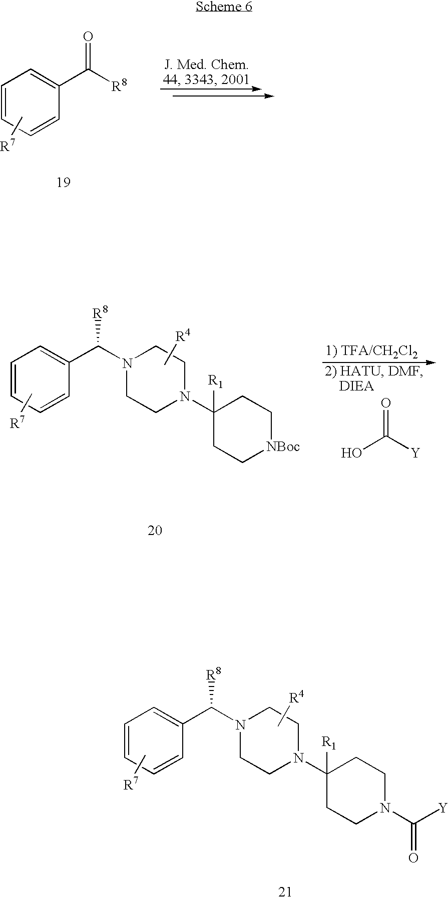

In Scheme 6, intermediate 20 was prepared from 19 according to the known procedure (J. Med. Chem., 44, 3343, 2001). Deprotection of 20 with TFA, followed by coupling with acids using HATU as an activator afforded 21.

### EXAMPLES

### Example 1

### Preparation of 8-methyl-4-quinolinecarboxylic acid

4-Chloro-8-methylquinoline was prepared from the reaction of 2-methylaniline with diethyl ethoxymethylenemalonate according to the known procedure (J. Heter. Chem., 34, 315, 1997). In a dried flask, a mixture of 4-chloro-8-methylquinoline (3 g, 17 mmol), Zn(CN)₂ (2.4 g, 20 mmol), and Pd(PPh₃)₄ (2.7 g, 2.6 mmol) in DMF (5 mL) was stirred at 110 to 120° C. for 4 h under N₂. After cooling to room temperature, the reaction mixture was poured into a chilled aqueous solution of NaHCO₃(10%, 40 mL). The solid was filtered off, and washed with EtOAc. The aqueous phase was extracted with EtOAc (3×50 mL), and the combined organic phase was dried over Na₂SO₄, and concentrated in vacuo. The residue was purified by flash chromatography (hexane-EtOAc, 95:5 to 85:15) to afford 8-methyl-4-quinolinecarbonitrile (2.0 g, 70%) as a white solid. ¹H NMR (CDCl₃) δ 2.82 (s, 3H), 7.65 (dd, 1H), 7.71 (m, 1H), 7.73 (d, 1H), 8.05 (m, 1H), 9.05 (d, 1H).

8-Methyl-4-quinolinecarbonitrile (1 g) was suspended in 50% KOH (5 mL) and ethylene glycol (15 mL). The mixture was kept at 160° C. for 24 h. After cooling to room temperature, the reaction mixture was poured into 20 mL 10% HCl solution. The solid was collected by filtration, washed with water, and dried to afford the title compound. ¹H NMR (DMSO-d₆/TFA): δ 2.70 (s, 3H), 7.55 (dd, 1H), 7.65 (m, 1H), 7.95 (d, 1H), 8.42 (m, 1H), 9.01 (d, 1H).

The following quinolinecarboxylic acids were prepared in a similar manner.

6-methyl-4-quinolinecarboxylic acid

7-chloro-4-quinolinecarboxylic acid

7-methyl-4-quinolinecarboxylic acid

2-methyl-4-quinolinecarboxylic acid

7-methoxy-4-quinolinecarboxylic acid

7-(2-hydroxyethoxy)-4-quinolinecarboxylic acid

5-quinolinecarboxylic acid

7-chloro-6-methyl-4-quinolinecarboxylic acid

7-trifluoromethyl4-quinolinecarboxylic acid

8-trifluoromethyl-4-quinolinecarboxylic acid

7-chloro-2-methyl-4-quinolinecarboxylic acid

### Example 2

### Preparation of 4-[(4-bromophenyl)(ethoxyimino)methyl]-1-(4-methyl-4-piperidinyl)-piperidine:

A mixture of 4-[4-(4-bromobenzoyl)-1-piperidinyl]-4-methyl-1-piperidinecarboxylic acid-1,1-diemthylethyl ester (10 g, 21.5 mmol), EtONH₂.HCl (8.3 g, 85 mmol), and sodium acetate (7 g, 85 mmol) in EtOH (150 mL) was heated at reflux for 6 h. After cooling to room temperature, the reaction mixture was quenched by addition of 4 N NaOH to pH12-13. The solvent was removed in vacuo, and the solid precipitated. The solid was collected by filtration, and re-dissolved in CH₂Cl₂ (400 mL). The organic phase was dried over Na₂SO₄, and concentrated in vacuo. The residue (11 g) was purified by flash chromatography (CH₂Cl₂-hexane-EtOAc, 12:3:1) to give 8Z (pure Z-isomer, 3.9 g), 8E (3.7 g, E-isomer), and Z/E mixture (2.1 g). To a stirred solution of 8Z (2.7 g, 5.3 mmol) in CH₂Cl₂ (15 mL) was added TFA (10 mL) at room temperature. After 2 h the reaction was concentrated, and the residue was redissolved in CH₂Cl₂ (150 mL). The organic phase was washed with 10% NaOH (2×25 mL) and brine (2×20 mL), dried over Na₂SO₄, and concentrated in vacuo. The crude product was purified by column chromatography to afford the title compound.

### Example 3

### Preparation of 4-[(4-bromophenyl)(2-pyridinyloxy)methyl]-1-(4-methyl-4-piperidinyl)piperidine:

To a stirred solution of 4-[(4-bromophenyl)hydroxymethyl]-1-piperidinyl]-4-methylpiperidinecarboxylic acid, 1,1-diemthylethyl ester (460 mg, 1 mmol), in DMF (4 mL, anhydrous) was added NaH (60% in mineral oil, 81 mg, 2.0 mmol) at room temperature. After 0.5 h., 2-fluoropyridine (262 mg, 2.7 mmol) was added, and the reaction was kept at 75° C. for 15 h. After cooling to room temperature, the reaction mixture was poured into ice water (20 mL). The reaction mixture was extracted with EtOAc (3×30 mL), washed with brine (2×10 mL), dried over Na₂SO₄, and concentrated in vacuo to afford crude product as a light yellow syrup. This residue was used in the next step without purification. A solution of the carbamate in TFA (5 mL) and CH₂Cl₂ (5 mL) was stirred at rt for 2 h, and was concentrated in vacuo. The residue was dissolved in CH₂Cl₂ (80 mL) and neutralized with 10%NaOH (30 mL). The reaction mixture was extracted with CH₂Cl₂(3×35 mL). The organic layers were combined, dried over Na₂SO₄, and concentrated in vacuo. The residue was purified by flash chromatography (CH₂Cl₂-MeOH-Et₃N, 100:5:0.1 to 70:30:0.1) to afford the title compound as a white amorphous solid (280 mg, 64%).

### Example 4

### Preparation of 4-[[4-[4-(4-bromobenzoyl)-1-piperidinyl]-4-methyl-1-piperidinyl]carbonyl]-7-chloroquinoline:

To a stirred solution of 4-[4-(4-bromobenzoyl)-1-piperidinyl]-4-methyl-1-piperidinecarboxylic acid1,1-diemthylethyl ester (400 mg, 0.86 mmol) in CH₂Cl₂ (6 mL) was added TFA (2 mL) at room temperature. After 2 h, the reaction was concentrated in vacuo, and dried under vacuum for 2 h. The residue is dissolved in DMF (5 mL) and 7-chloro-4-quinolinecarboxylic acid (214 mg, 1.03 mmol), HATU (490.5 mg, 1.29 mmol), and diisopropylethylamine (222 mg, 1.72 mmol) was added successively. After 16 h, the reaction was poured into ice water (15 mL), and extracted with EtOAc (3×30 mL). The organic phase was dried over Na₂SO₄, and concentrated in vacuo. The resulting residue was purified by column chromatography to afford the title compound. MS: 553.2 (M⁺−1). ¹H NMR (CDCl₃): δ0 0.96 (s, 3H), 1.24 (m, 1H), 1.52 (m, 1H), 1.66-1.96 (m, 5H), 2.10 (m, 1H), 2.18-2.32 (m, 2H), 2.84 (m, 1H), 2.92-3.14 (m, 2H), 3.20 (m, 1H), 3.28-3.54 (m, 2H), 4.36 (m, 1H), 7.32 (m, 1H), 7.56 (m, 1H), 7.61 and 7.80 (each m, 4H), 7.75 (m, 1H), 8.14(m, 1H), 8.95 (br.d, 1H).

### Example 5

### Preparation of 4-[1-(4-bromophenyl)ethenyl]-1-(4-methyl-4-piperidinyl)-piperidine

To a solution of CH₃PPh₃Br (1.7 g, 4.8 mmol) in THF (20 mL) was added n-BuLi (2 mL, 2.5 N in hexane, 5.0 mmol) at −40° C. The reaction was allowed to warm to 0° C., and stirred for 30 min at this temperature. A solution of 4-[4-(4-bromobenzoyl)-1-piperidinyl]-4-methyl-1-piperidinecarboxylic acid1,1-diemthylethyl ester (2 g, 4.3 mmol) in THF (15 mL) was added and stirred for 3 days. The mixture was poured into ice water and extracted with EtOAc (3×10 mL). The organic layers were washed with brine, and dried over Na₂SO₄. Concentration and purification by chromatography afforded the title compound.

### Example 6

### Preparation of 1-Hydroxy-4-[[4-methyl-4-[(3S)-3-methyl-4-[(1R)-1-[4-(trifluoromethyl)phenyl]ethyl]piperazinyl]-1-piperidinyl]carbonyl]quinolinium

To a solution of -4-[4-methyl-4-[(3S)-3-methyl-4-[(1R)-1-[4-(trifluoromethyl)phenyl]ethyl]piperazinyl]-1-piperidinecarboxylic aciddimethylethyl ester (234.4 mg, 0.5 mmol) in CH₂Cl₂ (5 mL) was added trifluoroacetic acid (2 mL) at room temperature. After 2 h the reaction mixture was concentrated in vacuo, and dried under vacuum. The residue is dissolved in DMF (6 mL) and 4-carboxy-1-hydroxyquinolinium (113.4 mg, 0.6 mmol), diisopropylethylamine (322 mg, 2.5 mmol), and HATU (285 mg, 0.75 mmol) was added successively at room temperature. After 16 h the reaction mixture was poured into ice water (15 mL), and the mixture was extracted with EtOAc (3×40 mL). The organic phase was washed with NaHCO₃ (15 mL, sat.) and brine (10 mL), and dried over Na₂SO₄. Concentration in vacuo, and purification by column chromatography (CH₂Cl₂-MeOH, 9:1) afforded the title compound as a white powder. MS: 539 (M⁺−1). ¹H NMR (CDCl₃, 400 MHz) δ 0.97 (s, 3H), 1.16 (br. d, 3H), 1.3 (dd, 3H), 1.52 (m, 1), 1.72 (m, 1H), 2.0 (t, 1H), 2.2 (m, 4H), 2.6 (dd, 1H), 3.1 (m, 1H), 3.41 (m, 1H), 3.6 (t, 1H), 4.0 (br. s, 1H), 4.3 (br. d, 1H), 7.22 (s, 1H), 7.58 (m, 4H), 7.76 (m, 1H), 7.8 (m, 1H), 7.9 (m, 1H), 8.53 (d, 1H), 8.8 (d, 1H).

### Example 7

### Preparation for 1-[[4-[4-[(4-Bromophenyl)(ethoxyimino)methyl]-1-piperidinyl]-4 methyl-1-piperidinyl]carbonyl]isoquinoline

To a solution of 4-[(4-bromophenyl)(ethoxyimino)methyl]-1-(4-methyl-4-piperidinyl)piperidine (50 mg, 0.12 mmol,), 1-isoquinolinecarboxylic acid (25 mg, 0.14 mmol), and Et₃N (44 mg, 0.43 mmol) in DMF (3 mL, anhydrous) was added HATU (60 mg, 0.16 mmol) at room temperature. After 16 h the reaction mixture was poured into ice water. The solid was collected by filtration and re-dissolved in CH₂Cl₂. The organic phase was dried over Na₂SO₄, and concentrated in vacuo. The residue was purified by preparative TLC (CH₂Cl₂-MeOH, 9:1) to afford the title compound as a white solid. MS: 564 (M⁺+1).

### Example 8

### Preparation of 3-[[4-[4-[(4-Bromophenyl)(ethoxyimino)methyl]-1-piperidinyl]-4-methyl-1-piperidinyl]carbonyl]isoquinoline

To a solution of oxime-amine, 4-[(4-bromophenyl)(ethoxyimino)methyl]-1-(4-methyl-4-piperidinyl)piperidine (50 mg, 0.12 mmol), 3-isoquinolinecarboxylic acid (25 mg, 0.14 mmol), and Et₃N (44 mg, 0.43 mmol) in DMF (3 mL, anhydrous) was added HATU (60 mg, 0.16 mmol) at room temperature. After 16 h the reaction mixture was poured into ice water. The solid was collected by filtration, and re-dissolved in CH₂Cl₂. The organic phase was dried over Na₂SO₄, and concentrated in vacuo. The residue was purified by preparative TLC (CH₂Cl₂-MeOH, 9:1) to afford the title compound as a light yellow powder. MS: 564 (M⁺+1).

### Example 9

### Preparation of 3-[[4-[4-[(4-Bromophenyl)(ethoxyimino)methyl]-1-piperidinyl]-4-methyl-1-piperidinyl]carbonyl]quinoline

To a stirred solution of 4-[(4-bromophenyl)(ethoxyimino)methyl]-1-(4-methyl-4-piperidinyl)piperidine (50 mg, 0.12 mmol), 3-quinolinecarboxylic acid (21 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and extracted with CH₂Cl₂ (3×10 mL). The organic phase was dried over Na₂SO₄, and concentrated in vacuo. The crude product was purified by preparative TLC to afford the title compound. MS: 563 (M⁺).

### Example 10

### Preparation of 2-[[4-[4-[(4-Bromophenyl)(ethoxyimino)methyl]-1-piperidinyl]-4-methyl-1-piperidinyl]carbonyl]quinoline

To a stirred solution of 4-[(4-bromophenyl)(ethoxyimino)methyl]-1-(4-methyl-4-piperidinyl)piperidine (50 mg, 0.12 mmol), 2-quinolinecarboxylic acid (21 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and extracted with CH₂Cl₂ (3×10 mL). The organic phase was dried over Na₂SO₄, and concentrated in vacuo., The crude product was purified by preparative TLC to afford the title compound.

### Example 11

### Preparation of 4-[[4-[4-[(4-Bromophenyl)(ethoxyimino)methyl]-1-piperidinyl]-4-methyl-1-piperidinyl]carbonyl]-2-methyl-3-quinolinol

To a stirred solution of 4-[(4-bromophenyl)(ethoxyimino)methyl]-1-(4-methyl-4-piperidinyl)piperidine (50 mg, 0.12 mmol), 3-hydroxy-2-methyl-4-quinoline carboxylic acid (27 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and was extracted with CH₂Cl₂ (3×10 mL). The organic phase was dried over Na₂SO₄, and concentrated in vacuo. The crude product was purified by preparative TLC to afford the title compound. MS: 593 (M⁺).

### Example 12

### Preparation of 4-[[4-[4-[(4-Bromophenyl)(ethoxyimino)methyl]-1-piperidinyl]-4-methyl-1-piperidinyl]carbonyl]-8-methylquinoline

To a stirred solution of 4-[(4-bromophenyl)(ethoxyimino)methyl]-1-(4-methyl-4-piperidinyl)piperidine (50 mg, 0.12 mmol), 8-methyl-4-quinoline carboxylic acid (25 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and was extracted with CH₂Cl₂ (3×10 mL). The organic phase was dried over Na₂SO₄, and concentrated in vacuo. The crude product was purified by preparative TLC to afford the title compound. MS: 577 (M⁺).

### Example 13

### Preparation of 4-[[4-[4-[(4-Bromophenyl)(ethoxyimino)methyl]-1-piperidinyl]-4-methyl-1-piperidinyl]carbonyl]-6-methylquinoline

To a stirred solution of 4-[(4-bromophenyl)(ethoxyimino)methyl]-1-(4-methyl-4-piperidinyl)piperidine (50 mg, 0.12 mmol), 6-methyl-4-quinoline carboxylic acid (25 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and was extracted with CH₂Cl₂ (3×10 mL). The organic phase was dried over Na₂SO₄, and concentrated in vacuo. The crude product was purified by preparative TLC to afford the title compound. MS: 577 (M⁺).

### Example 14

### Preparation of 2-[[4-[4-[(4-Bromophenyl)(ethoxyimino)methyl]-1-piperidinyl]-4-methyl-1-piperidinyl]carbonyl]-4-quinolinol

To a solution of 4-[(4-bromophenyl)(ethoxyimino)methyl]-1-(4-methyl-4-piperidinyl)piperidine (100 mg, 0.24 mmol), 4-hydroxy-2-quinolinecarboxylic acid(56 mg, 0.29 mmol) and Et₃N (87 mg, 0.86 mmol) in DMF (6 mL), HATU (119 mg, 0.31 mmol) was added at room temperature and the reaction was stirred for 24 h. The reaction mixture was poured into ice water and the first crop of solid was collected by filtration. The water layer was extracted with ethyl acetate and the organic layer was washed with NaHCO₃, dried and concentrated to give the second crop of solid. The two crops of crude were combined and purified by flash chromatography (2% to 10%, MeOH/CH₂Cl₂) to afford the title compound as an off white solid. MS 578 (M⁺).

### Example 15

### Preparation of 2-[[4-[4-[(4-Bromophenyl)(ethoxyimino)methyl]-1-piperidinyl]-4-methyl-1-piperidinyl]carbonyl]-4,8-quinolinediol

To a solution of 4-[(4-bromophenyl)(ethoxyimino)methyl]-1-(4-methyl-4-piperidinyl)piperidine (100 mg, 0.24 mmol), 4,8-dihydroxyquinoline-2-carboxylic acid (62 mg, 0.30 mmol) and Et₃N (87 mg, 0.86 mmol) in DMF (6 mL), HATU (119 mg, 0.31 mmol) was added at room temperature and the reaction was stirred for 24 h. The reaction mixture was poured into ice water and the first crop of solid was collected by filtration. The water layer was extracted with ethyl acetate and the organic layer was washed with NaHCO₃, dried and concentrated to give the second crop of solid. The two portions of crude were combined and purified by flash chromatography (2% to 10%, MeOH/CH₂Cl₂) to afford the title compound as a yellow solid. MS 594 (M⁺).

### Example 16

### Preparation of 2-[[4-[4-[(4-Bromophenyl)(ethoxyimino)methyl]-1-piperidinyl]-4-methyl-1-piperidinyl]carbonyl]-4-methoxyquinoline

To a solution of 4-[(4-bromophenyl)(ethoxyimino)methyl]-1-(4-methyl-4-piperidinyl)piperidine (100 mg, 0.24 mmol), 4-methoxy-2-quinolinecarboxylic acid (60 mg, 0.29 mmol) and Et₃N (87 mg, 0.86 mmol) in DMF (6 mL), HATU(119 mg, 0.31 mmol) was added at room temperature and the reaction was stirred for 24 h. The reaction mixture was poured into ice water and the first crop of solid was collected by filtration. The water layer was extracted with ethyl acetate and the organic layer was washed with NaHCO₃, dried and concentrated to give second crop of solid. The two crops of crude were combined and purified by flash chromatography (2% to 10%, MeOH/CH₂Cl₂) to afford the title compound as an off white solid. MS 592 (M⁺).

### Example 17

### Preparation of 4-[[4-[4-[(E)-(4-Bromophenyl)(ethoxyimino)methyl]-1-piperidinyl]-4-methyl-1-piperidinyl]carbonyl]-6-methyl-5-quinolinol

To a stirred solution of oxime-amine 4-[(E)-(4-bromophenyl)(ethoxyimino)methyl]-1-(4-methyl-4-piperidinyl)piperidine (50 mg, 0.12 mmol), 5-hydroxy-6-methyl-4-quinolinecarboxylic acid (28 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and the solid was collected by filtration. Purification by preparative TLC afforded the title compound. ¹H NMR (CDCl₃, 400 MHz) δ 0.9 (d, 3H), 1.2 (t, 3H), 1.36-2.04 (m, 6H), 2.06-2.34 (m, 4H), 2.9-3.1 (m, 2H), 3.1-3.36 (m, 2H), 3.38-3.65 (m, 2H), 3.90-4.14 (m, 1H), 4.15-4.30 (q, 2H), 7.02-7.16 (m, 2H), 7.2-7.36 (m, 2H), 7.44-7.56 (m, 2H), 7.64-7.76 (s, 1H), 8.10-8.20 (d, 1H).

### Example 18

### Preparation of 4-[[4-[4-[(E)-(4-Bromophenyl)(ethoxyimino)methyl]-1-piperidinyl]-4-methyl-1-piperidinyl]carbonyl]-7-chloro-6-methylquinoline

To a stirred solution of 4-[(4-bromophenyl)(ethoxyimino)methyl]-1-(4-methyl-4-piperidinyl)piperidine (50 mg, 0.12 mmol), 7-chloro-6-methyl-4-quinolinecarboxylic acid (30 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and the solid was collected by filtration. The solid was redissolved in CH₂Cl₂, and purified by flash chromatography to afford the title compound. ¹H NMR (CDCl₃, 400 MHz) δ 0.9 (d, 3H), 1.2 (t, 3H), 1.36-1.77 (m, 7H), 1.77-1.88 (m, 1H), 1.94-2.3 (m, 3H), 2.5 (s, 3H), 2.7-2.86 (m, 1H), 2.88-3.08 (m, 2H), 3.1-3.4 (m, 2H), 3.44-3.7 (m, 1H) 4.15-4.30 (q, 2H), 7.02-7.3 (m, 3H), 7.38-7.58 (m, 2H), 7.59-7.8 (d, 1H), 8.1-8.3 (s, 1H), 8.70-9.0 (m, 1H).

### Example 19

### Preparation of 3-[[4-[4-[(E)-(4-Bromophenyl)(ethoxyimino)methyl]-1-piperidinyl]-4-methyl-1-piperidinyl]carbonyl]-6-(trifluoromethyl)-7-quinolinol and, 3-[[4-[4-[(Z)-(4-Bromophenyl)(ethoxyimino)methyl]-1-piperidinyl]-4-methyl-1-piperidinyl]carbonyl]-6-(trifluoromethyl)-7-quinolinol

To a stirred solution of 4-[(Z)-(4-bromophenyl)(ethoxyimino)methyl]-1-(4-methyl-4-piperidinyl)piperidine (50 mg, 0.12 mmol), 7-hydroxy-6-(trifluoromethyl)-3-quinolinecarboxylic acid (34 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and solid was collected by filtration. The solid was dissolved in CH₂Cl₂, and purified by preparative TLC to afford E-isomer and Z-isomer E-isomer, ¹H NMR (CDCl₃, 400 MHz) δ 0.93 (s, 3H), 1.2 (t, 3H), 1.24-2.7 (m, 10H), 2.8-3.36 (m, 4H), 3.38-4.16 (m, 3H), 3.18-4.24 (q, 2H), 7.08-7.18 (m, 2H), 7.4-7.58 (m, 3H), 7.59-7.7 (m, 1H), 7.78-7.9 (m, 1H), 8.5-8.62 (s, 1H), and Z-isomer.

¹H NMR (CDCl₃) 400 MHz) δ 0.93 (s, 3H), 1.2 (t, 3H), 1.24-2.7 (m, 12H), 2.8-3.4 (m, 3H), 3.5-3.9 (m, 2H), 3.96-4.14 (q, 2H), 7.08-7.18 (m, 2H), 7.48-7.55 (m, 2H), 7.55-7.64 (m, 1 H), 7.68-7.78 (m, 1H), 7.89-7.98 (s, 1H), 8.52-8.62 (s, 1H).

### Example 20

### Preparation of 3-[[4-[4-[(4-Bromophenyl)(ethoxyimino)methyl]-1-piperidinyl]-4-methyl-1-piperidinyl]carbonyl]-8-(trifluoromethyl)-4-quinolinol

To a stirred solution of 4-[(4-bromophenyl)(ethoxyimino)methyl]-1-(4-methyl-4-piperidinyl)piperidine (50 mg, 0.12 mmol), 4-hydroxy-8-trifluoromethyl-3-quinolinecarboxylic acid (28 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and extracted with CH₂Cl₂ (3×10 mL). The organic phase was dried over Na₂SO₄, and concentrated in vacuo. The crude product was purified by preparative TLC to afford the title compound.

### Example 21

### Preparation of 2-[[4-[4-[(4-Bromophenyl)(ethoxyimino)methyl]-1-piperidinyl]-4-methyl-1-piperidinyl]carbonyl]-6-ethyl-4-quinolinol

To a stirred solution of 4-[(4-bromophenyl)(ethoxyimino)methyl]-1-(4-methyl-4-piperidinyl)-piperidine (50 mg, 0.12 mmol), 6-ethyl-4-hydroxy-2-quinolinecarboxylic acid (28 mg, 0.13 mmol), and Et₃N (24 mg, 0.24 mmol) in DMF (2 mL), HATU (61 mg, 0.16 mmol) was added at room temperature. After 16 h the mixture was poured into ice water (10 mL) and extracted with CH₂Cl₂ (3×10 mL). The organic phase was dried over Na₂SO₄, and concentrated in vacuo. The crude product was purified by preparative TLC to afford the title compound.

### Example 22

### Preparation of 3-[[4-[4-[(4-Bromophenyl)(ethoxyimino)methyl]-1-piperidinyl]-4-methyl-1-piperidinyl]carbonyl]-7-(trifluoromethyl)-4-quinolinol

To a solution of 4-[(4-bromophenyl)(ethoxyimino)methyl]-1-(4-methyl-4-piperidinyl)piperidine (100 mg, 0.24 mmol), 4-hydroxy-7-trifluoromethyl-3-quinolinecarboxylic acid (87 mg, 0.34 mmol) and Et₃N (87 mg, 0.86 mmol) in DMF(6 mL), HATU(119 mg, 0.31 mmol) was added at room temperature and the reaction was stirred for 24 hours. The reaction mixture was poured into ice water and the first crop of solid was collected by filtration. The water layer was extracted with ethyl acetate and the organic layer was washed with NaHCO₃, dried and concentrated to give the second crop of solid. The two crops of crude were combined and purified by flash chromatography (2% to 10%, MeOH/CH₂Cl₂) to afford the title compound as an off-white solid. MS 646 (M⁺).

### Example 23

### Preparation of 2-[[4-[4-[(4-Bromophenyl)(ethoxyimino)methyl]-1-piperidinyl]-4-methyl-1-piperidinyl]carbonyl]-8-methyl-4-quinolinol

To a stirred solution of 4-[(4-bromophenyl)(ethoxyimino)methyl]-1-(4-methyl-4-piperidinyl)piperidine (50 mg, 0.12 mmol), 4-hydroxy-8-methyl-2-quinoline carboxylic acid (28 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and extracted with CH₂Cl₂ (3×10 mL). The organic phase was dried over Na₂SO₄, and concentrated in vacuo. The crude product was purified by preparative TLC to afford the title compound.

### Example 24

### Preparation of 4-[[4-[4-[(4-Bromophenyl)(ethoxyimino)methyl]-1-piperidinyl]-4-methyl-1-piperidinyl]carbonyl]-2-phenylquinoline

To a solution of 4-[(4-bromophenyl)(ethoxyimino)methyl]-1-(4-methyl-4-piperidinyl)piperidine (100 mg, 0.24 mmol), 2-phenyl-4-quinolinecarboxylic acid (73 mg, 0.29 mmol) and Et₃N (87 mg, 0.86 mmol) in DMF(6 mL), HATU(119 mg, 0.31 mmol) was added at room temperature and the reaction was stirred for 24 h. The reaction mixture was poured into ice water and the first crop of solid was collected by filtration. The water layer was extracted with ethyl acetate and the organic layer was washed with NaHCO₃, dried and concentrated to give the second crop of solid. The two crops of crude were combined and purified by flash chromatography (2% to 10%, MeOH/CH₂Cl₂) to afford the title compound as a light orange solid. MS 638 (M⁺).

### Example 25

### Preparation of 6-[[4-[4-[(E)-(4-Bromophenyl)(ethoxyimino)methyl]-1-piperidinyl]-4-methyl-1-piperidinyl]carbonyl]quinoline

To a solution of 4-[(4-bromophenyl)(ethoxyimino)methyl]-1-(4-methyl-4-piperidinyl)piperidine (50 mg, 0.12 mmol), quinoline-6-carboxylic acid (25 mg, 0.14 mmol), and Et₃N (44 mg, 0.43 mmol) in DMF (3 mL, anhydrous) was added HATU (60 mg, 0.16 mmol) at room temperature. After 16 h the reaction mixture was poured into ice water, and the solid was collected by filtration. The solid was dissolved in CH₂Cl₂, and dried over Na₂SO₄. Concentration in vacuo, and purification by preparative TLC (CH₂Cl₂-MeOH, 9:1) afforded the title compound as a white powder. MS: 564 (M⁺−1).

### Example 26

### Preparation of 2-[[4-[4-[(4-Bromophenyl)(ethoxyimino)methyl]-1-piperidinyl]-4-methyl-1-piperidinyl]carbonyl]-7-ethyl-4-quinolinol

To a stirred solution of 4-[(4-bromophenyl)(ethoxyimino)methyl]-1-(4-methyl-4-piperidinyl)piperidine (50 mg, 0.12 mmol), 7-ethyl-4-hydroxy-2-quinolinecarboxylic acid (28 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and was extracted with CH₂Cl₂ (3×10 mL). The organic phase was dried over Na₂SO₄, and concentrated in vacuo. The crude product was purified by preparative TLC to afford the title compound. MS: 606 (M⁺−1).

### Example 27

### Preparation of 4-[[4-[4-[(Z)-(4-Bromophenyl)(ethoxyimino)methyl]-1-piperidinyl]-4-methyl-1-piperidinyl]carbonyl]quinoline

To a solution of 4-[(Z)-(4-bromophenyl)(ethoxyimino)methyl]-1-(4-methyl-4-piperidinyl)piperidine (240 mg, 0.59 mmol), quinoline-4-carboxylic acid (112 mg, 0.64 mmol) and Et₃N (119 mg, 1.18 mmol) in DMF (2 mL), HATU (290 mg, 0.76 mmol) was added at room temperature. After 16 h, the reaction mixture was poured into ice water, and the solid was collected by filtration. Further purification by flash chromatography afforded title compound. ¹H NMR (DMSO-d₆) δ 0.9 (s, 3H), 1.18 (t, 3H), 1.22-1.85 (m, 7H), 1.98-2.18 (m, 3H), 2.39 (m, 1H), 2.75 (m, 1H), 2.96 (m, 2H), 3.31 (q, 1H), 3.50 (q, 1H), 4.04 (q, 2H), 4.26 (m, 1H), 7.09 (m, 2H), 7.28 (m, 1H), 7.5 (m, 2H), 7.58 (q, 1H), 7.71-7.85 (m, 2H), 8.13 (d, 1H), 8.92 (d, 1H).

### Example 28

### Preparation of 4-[[4-[4-[(4-Bromophenyl)(ethoxyimino)methyl]-1-piperidinyl]-4-methyl-1-piperidinyl]carbonyl]-7-(trifluoromethyl)quinoline

To a solution of 4-[(4-bromophenyl)(ethoxyimino)methyl]-1-(4-methyl-4-piperidinyl)piperidine (50 mg, 0.12 mmol), a mixture of 7-trifluoromethyl-4-quinolinecarboxylic acid (25 mg, 0.14 mmol), Et₃N (0.06 mL, 0.43 mmol) in DMF (3 mL, anhydrous) was added HATU(60 mg, 0.16 mmol) at room temperature. After 16 h the reaction mixture was poured into ice water, and the solid was collected by filtration. The solid was dissolved in CH₂Cl₂; and dried over Na₂SO₄. Concentration in vacuo, and purification by preparative TLC (CH₂Cl₂-MeOH, 9:1) afforded the title compound as a light yellow powder. MS. 630 (M⁺−1).

### Example 29

### Preparation of 4-[[4-[4-[(Z)-(4-Bromophenyl)(ethoxyimino)methyl]-1-piperidinyl]-4-methyl-1-piperidinyl]carbonyl]-1-hydroxyquinolinium

To a solution of 4-[(Z)-(4-bromophenyl)(ethoxyimino)methyl]-1-(4-methyl-4-piperidinyl)piperidine (120 mg, 0.29 mmol), 4-carboxylic-1-hydroxyquinolinium (61 mg, 0.32 mmol), and Et₃N (59 mg, 0.58 mmol) in DMF (2 mL), HATU (145 mg, 0.38 mmol) was added at room temperature. After 16 h the reaction mixture was poured into ice water and filtered. The solid was dissolved in CH₂Cl₂ (2 mL) and purified by flash chromatography to afford title compound as light yellow solid. MS: 579 (M+). ¹H NMR (CDCl₃, 400 MHz) δ 0.9 (s, 3H), 1.2 (t, 3H), 1.21-1.84 (m, 7H), 1.95-2.2 (m, 3H), 2.38-2.5 (m, 1H), 2.75-2.82 (m, 1H), 2.9-3.08 (m,1H), 3.3-3.6 (m, 2H), 4.04 (q, 2H), 4.25-4.40 (m, 1H), 7.08-7.14 (m, 2H), 7.18-7.25 (m, 1H), 7.5 (m, 2H),7.65-7.75 (m, 1H),7.78-7.96 (m, 2H).

### Example 30

### Preparation of 7-[[4-[4-[(Z)-(4-Bromophenyl)(ethoxyimino)methyl]-1-piperidinyl]-4-methyl-1-piperidinyl]carbonyl]quinoline

To a solution of 4-[(Z)-(4-bromophenyl)(ethoxyimino)methyl]-1-(4-methyl-4-piperidinyl)piperidine (50 mg, 0.12 mmol), quinoline-7-carboxylic acid (25 mg, 0.14 mmol), and Et₃N (44 mg, 0.43 mmol) in DMF (3 mL) was added HATU (60 mg, 0.16 mmol) at room temperature. After 16 h, the reaction mixture was poured into ice water. The solid was collected by filtration, and was re-dissolved in CH₂Cl₂, and dried over Na₂SO₄. Concentration and purification by preparative TLC (CH₂Cl₂-MeOH, 9:1) afforded the title compound as a brown powder. MS: 562.1(M⁺−1). ¹H NMR (CDCl₃) δ 0.93 (s, 3H), 1.20 (t, 3H), 1.31-1.84 (m, 7H), 1.98 (br.d, 1H), 2.06-2.18 (m, 2H), 2.42 (tt, 1H), 2.84 (br. d, 1H), 2.99(m, 1H), 3.3-3.42 (m, 1H), 3.52 (br.t, 2H), 4.06 (q, 2H), 4.12 (m, 1H), 7.09-7.13 (m, 2H), 7.45 (dd, 1H), 7.51-7.54 (m, 2H), 7.59 (dd, 1H),7.86 (d, 1H), 8.09 (d, 1H), 8.18 (dd, 1H), 8.96 (dd, 1H).

### Example 31

### Preparation of 8-[[4-[4-[(Z)-(4-Bromophenyl)(ethoxyimino)methyl]-1-piperidinyl]-4-methyl-1-piperidinyl]carbonyl]quinoline

To a stirred solution of 4-[(Z)-(4-bromophenyl)(ethoxyimino)methyl]-1-(4-methyl-4-piperidinyl)piperidine (150 mg, 0.37 mmol), 8-quinolinecarboxylic acid (70 mg, 0.41 mmol), and Et₃N (75 mg, 0.73 mmol) in DMF (5 mL), HATU (183 mg, 0.48 mmol) was added at room temperature. After 16 h the mixture was poured into ice water and filtered. The solid was dissolved in CH₂Cl₂ (2 mL) and purified by flash chromatography to afford the title compound as a white solid. MS:563 (M⁺). ¹H-NMR (CDCl₃, 400 MHz) δ 0.94 (d, 3H), 1.2 (m, 3H), 1.24-2.0 (m, 7H), 2.0-2.2 (m, 3H), 2.3-2.5 (m, 1H), 2.7-2.82 (m, 1H), 2.9-3.08 (m, 2H), 3.2-3.8 (m, 2H), 4.04 (m, 2H), 4.1-4.40 (m, 1H), 7.08-7.14 (d, 2H), 7.39-7.45 (m, 1H), 7.48-7.58 (m, 3H), 7.62-7.68 (m, 1H),7.8-7.86 (m, 1H), 8.13-8.18 (m, 1H), 8.9-9.0 (m, 1H).

### Example 32

### Preparation of 4-[[4-[4-[(Z)-(4-Bromophenyl)(ethoxyimino)methyl]-1-piperidinyl]-4-methyl-1-piperidinyl]carbonyl]-7-chloroquinoline

To a stirred solution of 4-[(Z)-(4-bromophenyl)(ethoxyimino)methyl]-1-(4-methyl-4-piperidinyl)piperidine (50 mg, 0.12 mmol), 7-chloro-4-quinolinecarboxylic acid (28 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water and filtered. The solid was redissolved in CH₂Cl₂ (2 mL) and purified by preparative TLC to afford title compound as a yellow oil. ¹H-NMR (CDCl₃, 400 MHz) δ 0.93 (s, 3H), 1.2 (t, 3H), 1.24-1.96 (m, 7H), 1.98-2.2 (m, 3H), 2.36-2.5 (m, 1H), 2.72-2.84 (m, 1H), 2.86-3.06 (m, 2H), 3.24-3.6 (m, 2H), 4.04 (q, 2H), 4.15-4.34 (m, 1H), 7.08-7.14 (m, 2H), 7.28-7.33 (m, 1H), 7.40-7.62 (m, 3H), 7.71-7.82 (m, 1H), 8.1-8.18 (d, 1H), 8.9-8.98 (d, 1H).

### Example 33

### Preparation of 4-[[4-[4-[(Z)-(4-Bromophenyl)(ethoxyimino)methyl]-1-piperidinyl]-4-methyl-1-piperidinyl]carbonyl]-7-methylquinoline

To a stirred solution of 4-[(Z)-(4-bromophenyl)(ethoxyimino)methyl]-1-(4-methyl-4-piperidinyl)piperidine (50 mg, 0.12 mmol), 7-methyl-4-quinolinecarboxylic acid (25 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water and filtered. The solid was dissolved in CH₂Cl₂ (2 mL) and purified by preparative TLC to afford title compound as a yellow oil. ¹H-NMR(CDCl₃, 400 MHz) δ 0.94 (s, 3H), 1.2 (t, 3H), 1.24-1.88 (m, 7H), 1.96-2.18 (m, 3H), 2.36-2.46 (m, 1H), 2.54-2.61 (d, 3H), 2.72-2.81 (m, 1H), 2.9-3.3 (m, 2H), 3.24-3.38 (m,1H),3.46-3.58 (m,1H), 4.06 (q, 2H), 4.16-4.32 (m, 1H), 7.08-7.14 (dd, 2H), 7.20-7.25 (m, 1H), 7.40-7.46 (m, 1H),7.49-7.54 (m, 2H),7.66-7.76 (m,1H), 7.9-7.93 (s, 1H), 8.90 (m, 1H).

### Example 34

### Preparation of 4-[[4-[4-[(E)-(4-Bromophenyl)(ethoxyimino)methyl]-1-piperidinyl]-4-methyl-1-piperidinyl]carbonyl]-7-chloro-1-hydroxyquinolinium

To a stirred solution of 4-[(E)-(4-bromophenyl)(ethoxyimino)methyl]-1-(4-methyl-4-piperidinyl)piperidine (50 mg, 0.12 mmol), 4-carboxy-7-chloro-1-hydroxy-quiniolinium (30 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water and filtered. The solid was dissolved in CH₂Cl₂ (2 mL) and purified by preparative TLC to afford title compound as a light yellow solid. MS: 614 (M⁺+1)). ¹H-NMR(CDCl₃, 400 MHz) δ 0.93 (s, 3H), 1.2 (t, 3H), 1.24-1.96 (m, 7H), 1.98-2.2 (m, 3H), 2.36-2.5 (m, 1H), 2.74-2.84 (m, 1H), 2.93-3.08 (m, 2H), 3.2-3.7 (m, 2H), 4.04 (q, 2H), 4.15-4.30 (m, 1H), 7.08-7.14 (m, 2H), 7.18-7.26 (m, 1H), 7.48-7.56 (m, 2H), 7.61-7.68 (m, 1H), 7.76-7.9 (m, 1H), 8.5 (d, 1H), 8.8 (d,1H).

### Example 35

### Preparation of 8-[[4-[4-[(Z)-(4-Bromophenyl)(ethoxyimino)methyl]-1-piperidinyl]4-methyl-1-piperidinyl]carbonyl]4-chloroquinoline

To a stirred solution of 4-[(Z)-(4-bromophenyl)(ethoxyimino)methyl]-1-(4-methyl-4-piperidinyl)piperidine (50 mg, 0.12 mmol), 4-chloro-8-quinolinecarboxylic acid (30 mg, 0.14 mmol), Et₃N (44 mg, 0.43 mmol) in DMF (3 mL) was added HATU (60 mg, 0.16 mmol) at room temperature. After 16 h, the reaction mixture was poured into ice water while stirring vigorously. The solid was collected by filtration, and was re-dissolved in CH₂Cl₂ and dried over Na₂SO₄. Concentration and purification by preparative TLC (CH₂Cl₂-MeOH, 9:1) afforded product as a white powder. LC-MS: 596 (M⁺−1); ¹H NMR (CDCl₃) δ 0.91 (d, 3H), 1.16-1.22 (m, 3H), 1.25-2.14 (m, 10H), 2.39 (m, 1H), 2.78 (m, 1H), 2.90 (m, 1H), 3.00 (m, 1H), 3.28 (m, 1H), 3.50-3.65 (m, 1H), 4.05 (m, 2H), 4.18-4.35 (m, 1H), 7.09-7.11 (m, 2H), 7.49-7.54 (m, 3H), 7.64-7.72 (m, 2H), 8.24-8.27 (m, 1H), 8.79-8.82 (m, 1H).

### Example 36

### Preparation of 7-[[4-[4-[(Z)-(4-Bromophenyl)(ethoxyimino)methyl]-1-piperidinyl]4-methyl-1-piperidinyl]carbonyl]4-chloroquinoline

To a solution of 4-[(Z)-(4-bromophenyl)(ethoxyimino)methyl]-1-(4-methyl-4-piperidinyl)piperidine (50 mg, 0.12 mmol), 4-chloro-7-quinolinecarboxylic acid (50 mg, 0.24 mmol), and Et₃N(0.12 mL, 0.86 mmol) in DMF (3 mL) was added HATU(120 mg, 0.32 mmol) at room temperature. After 16 h, the reaction mixture was poured into ice water while stirring vigorously. The solid was collected, and re-dissolved in CH₂Cl₂ and dried over Na₂SO₄. Concentration and purification by preparative TLC(CH₂Cl₂-MeOH, 9:1) afforded the title compound as a light yellow powder.

LC-MS: 596 (M⁺−1). ¹H NMR (CDCl₃) δ 0.93 (d, 3H), 1.20 (t, 3H), 1.26-1.80 (m, 7H), 1.98-2.17 (m, 3H), 2.42 (tt, 1H), 2.83 (br. d, 1H), 2.99 (br.d, 1H), 3.33 (m, 1H), 3.51 (br.t, 2H), 4.06 (q, 2H), 4.13 (m, 1H), 7.11 (m, 2H), 7.51-7.54 (m, 3H), 7.69 (dd, 1H), 8.11 (d, 1H), 8.28 (d, 1H), 8.82 (d, 1H).

### Example 37

### Preparation of 4-[[4-[4-[(Z)-(4-Bromophenyl)(ethoxyimino)methyl]-1-piperidinyl]-4-methyl-1-piperidinyl]carbonyl]-2-methylquinoline

To a stirred solution of 4-[(Z)-(4-bromophenyl)(ethoxyimino)methyl]-1-(4-methyl-4-piperidinyl)piperidine (50 mg, 0.12 mmol), 2-methyl-4-quinolinecarboxylic acid (25 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water and filtered. The solid was dissolved in CH₂Cl₂ (2 mL) and purified by flash chromatography to afford title compound as a colorless oil. ¹H NMR (CDCl₃, 400 MHz) δ 0.94(d, 3H), 1.2 (t, 3H), 1.24-1.88 (m, 7H), 1.96-2.18 (m, 3H), 2.36-2.46 (m, 1H), 2.72-2.82 (m, 4H), 2.92-3.03 (m, 2H), 3.24-3.6 (m, 2H), 4.06 (q, 2H), 4.16-4.3 (m, 1H), 7.08-7.14 (dd, 2H), 7.18-7.24 (m, 1H), 7.48-7.58 (m, 3H), 7.68-7.8 (m, 2H), 8.02-8.06 (d, 1H).

### Example 38

### Preparation of 5-[[4-[4-[(Z)-(4-Bromophenyl)(ethoxyimino)methyl]-1-piperidinyl]-4-methyl-1-piperidinyl]carbonyl]-1-hydroxyquinolinium

To a stirred solution of 4-[(Z)-(4-bromophenyl)(ethoxyimino)methyl]-1-(4-methyl-4-piperidinyl)piperidine (100 mg, 0.25 mmol), 5-carboxy-1-hydroxy-quinolinium (51 mg, 0.28 mmol), and Et₃N (51 mg, 0.5 mmol) in DMF (2 mL), HATU (124 mg, 0.33 mmol) was added at room temperature. After 16 h the mixture was poured into ice water and filtered. The solid was dissolved in CH₂Cl₂ (2 mL) and purified by flash chromatography to afford title compound as a light yellow solid. MS: 580 (M⁺−1). ¹H NMR(CDCl₃, 400 MHz) δ 0.94 (d, 3H), 1.2 (m, 3H), 1.24-1.94 (m, 7H), 1.96-2.2 (m, 3H), 2.36-2.5 (m, 1H), 2.7-2.84 (m, 1H), 2.9-3.08 (m, 2H), 3.2-3.6 (m, 2H), 4.04 (q, 2H), 4.1-4.38 (m, 1H), 7.08-7.14 (dd, 2H), 7.30-7.38 (m, 1H), 7.48-7.6 (m, 3H), 7.72-7.82 (m, 2H), 8.52-8.58 (d, 1H), 8.78-8.82 (d, 1H).

### Example 39

### Preparation of 4-[[4-[4-[(E)-(4-Bromophenyl)(ethoxyimino)methyl]-1-piperidinyl]-4 methyl-1-piperidinyl]carbonyl]-7-methoxyquinoline

To a stirred solution of 4-[(E)-(4-bromophenyl)(ethoxyimino)methyl]-1-(4-methyl-4-piperidinyl)piperidine (50 mg, 0.12 mmol), 7-methoxy-4-quinolinecarboxylic acid (27 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water and filtered. The solid was dissolved in CH₂Cl₂ (2 mL) and purified by HPLC to afford the title compound, trifluoroacetic acid salt, as a white solid. MS: 593 (M⁺). ¹H-NMR (CDCl₃, 400 MHz): δ 1.2 (m, 3H), 1.38-1.52 (m, 3H), 1.66-3.04 (m, 12H), 3.04-3.80 (m, 6H), 3.9-4.15 (m, 5H), 4.80-5.1 (m, 1H), 7.1-7.22 (m, 2H), 7.4-7.6 (m, 4H), 7.7-8.0 (m, 3H).

### Example 40

### Preparation of 5-[[4-[4-[(E)-(4-Bromophenyl)(ethoxyimino)methyl]-1-piperidinyl]-4-methyl-1-piperidinyl]carbonyl]quinoline

To a stirred solution of 4-[(E)-(4-bromophenyl)(ethoxyimino)methyl]-1-(4-methyl-4-piperidinyl)piperidine (150 mg, 0.37 mmol), 5-quinolinecarboxylic acid (70 mg, 0.4 mmol), and Et₃N (74 mg, 0.73 mmol) in DMF (10 mL), HATU (183 mg, 0.48 mmol) was added at room temperature. After 16 h the mixture was poured into ice water and filtered. The solid was dissolved in CH₂Cl₂ (2 mL) and purified by flash chromatography to afford the title compound as a brown solid. MS: 563 (M⁺). ¹H NMR (CDCl₃, 400 MHz) δ 0.9 (d, 3H), 1.2 (t, 3H), 1.2-1.84 (m, 7H), 1.95-2.2 (m, 3H), 2.3-2.5 (m, 1H), 2.7-2.82 (m, 1H), 2.9-3.08 (m, 2H), 3.2-3.6 (m, 2H), 4.04 (q, 2H), 4.25-4.40 (m, 1H), 7.08-7.14 (d, 2H), 7.4-7.5 (m, 4H), 7.7 (m, 1H), 8.1-8.3 (m, 2H), 8.9-9.0 (m, 1H).

### Example 41

### Preparation of 2-[[4-[[4-[4-[(Z)-(4-Bromophenyl)(ethoxyimino)methyl]-1-piperidinyl]-4-methyl-1-piperidinyl]carbonyl]-7-quinolinyl]oxy]ethanol

To a stirred solution of 4-[(Z)-(4-bromophenyl)(ethoxyimino)methyl]-1-(4-methyl-4-piperidinyl)piperidine (50 mg, 0.12 mmol), 7-(2-hydroxyethoxy)-4-quinolinecarboxylic acid (30 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water and filtered. The solid was dissolved in CH₂Cl₂ (2 mL), and purified by HPLC to afford title compound, trifluoroacetic acid salt, as a white solid. MS:622 (M⁺−1 ¹H NMR (CDCl₃, 400 MHz) δ 1.18-1.3 (m, 3H), 1.4-1.55 (m, 3H), 1.7-1.82 (m, 1H), 2.0-2.6 (m, 6H), 2.6-3.4 (m, 7H), 3.4-3.8 (m, 2H),3.9-5.0 (m, 4H), 6.80-7.2 (m, 4H), 7.4-7.6 (m, 3H), 7.7-8.0 (m, 2H), 8.8-9.1 (m, 1H).

### Example 42

### Preparation of 4-[[4-[4-[(Z)-(4-Bromophenyl)(ethoxyimino)methyl]-1-piperidinyl]-4-methyl-1-piperidinyl]carbonyl]-3-methylquinoline

To a stirred solution of 4-[(Z)-(4-bromophenyl)(ethoxyimino)methyl]-1-(4-methyl-4-piperidinyl)piperidine (100 mg, 0.25 mmol), 3-methyl-4-quinolinecarboxylic acid (51 mg, 0.28 mmol), and Et₃N (51 mg, 0.5 mmol) in DMF (2 mL), was added HATU (123.5 mg, 0.33 mmol) at room temperature. After 16 h the reaction mixture was poured into ice water and filtered. The solid was re-dissolved in CH₂Cl₂, and purified by flash chromatography to afford the title compound as a colorless oil. MS 577 (M⁺). ¹H NMR (CDCl₃) δ 0.9 (d, 3H), 1.2 (t, 3H), 1.4-1.84 (m, 7H), 2.0-2.2 (m, 3H), 2.38-2.5 (m, 4H), 2.73-3.05 (m, 3H), 3.2-3.4(m, 1H), 3.45-3.65 (m, 1H), 4.04 (q, 2H), 4.25-4.40 (m, 1H), 7.08-7.14 (m, 2H), 7.48-7.6 (m, 3H), 7.64-7.74 (m, 2H), 8.06-8.12 (d, 1H), 8.78-8.81 (d, 1H).

### Example 43

### Preparation of 8-[[4-[4-[(Z)-(4-Bromophenyl)(ethoxyimino)methyl]-1-piperidinyl]-4-methyl-1-piperidinyl]sulfonyl]quinoline

To a solution of 4-[(Z)-(4-bromophenyl)(ethoxyimino)methyl]-1-(4-methyl-4-piperidinyl)piperidine (50 mg, 0.12 mmol) and Et₃N (44 mg, 0.43 mmol) in CH₂Cl₂ (3 mL) was added 8-quinolinesulfonyl chloride (40 mg, 0.18 mmol) at room temperature. After 2 h the solvent was removed in vacuo, and the resulting residue was purified by preparative TLC (CH₂Cl₂-MeOH, 9:1) to afford title compound as a light yellow powder. ¹H NMR (CDCl₃) δ 0.82 (s, 3H), 1.18 (t, 3H), 1.23-1.46 (m, 4H), 1.68 (br.d, 2H), 1.8-1.9 (m, 2H), 2.20 (br.d, 2H), 2.34 (tt, 1H), 2.83 (br.d, 2H), 3.34-3.44 (m, 2H), 3.50-3.58 (m, 2H), 4.03 (q, 2H), 7.14 (d, 2H), 7.46-7.52 (m, 3H), 7.59 (t, 1H), 8.02 (dd, 1H), 8.24 (dd, 1H), 8.45 (dd, 1H), 9.04 (dd, 1H).

### Example 44

### Preparation of 4-[[4-[4-[(4-Bromophenyl)(ethoxyimino)methyl]-1-piperidinyl]-4-methyl-1-piperidinyl]carbonyl]cinnoline

To a solution of 4-[(Z)-(4-bromophenyl)(ethoxyimino)methyl]-1-(4-methyl-4-piperidinyl)piperidine (50 mg, 0.12 mmol), cinnoline-4-carboxylic acid (25 mg, 0.14 mmol), Et₃N (0.06 mL, 0.43 mmol) in DMF (3 mL, anhydrous) was added HATU (60 mg, 0.16 mmol) at room temperature. After 16 h the reaction mixture was poured into ice water, and the solid was collected by filtration. The solid was dissolved in CH₂Cl₂, and dried over Na₂SO₄. Concentration in vacuo, and purification by preparative TLC(CH₂Cl₂-MeOH, 9:1) afforded the title compound as a light yellow powder. MS: 564 (M⁺).

### Example 45

### Preparation of 2-[[4-[4-[(4-Bromophenyl)(ethoxyimino)methyl]-1-piperidinyl]-4-methyl-1-piperidinyl]carbonyl]quinoxaline

To a solution of 4-[(4-bromophenyl)(ethoxyimino)methyl]-1-(4-methyl-4-piperidinyl)piperidine (50 mg, 0.12 mmol), 2-quinoxalinecarboxylic acid (25 mg, 0.14 mmol), Et₃N (0.06 mL, 0.43 mmol) in DMF (3 mL, anhydrous) was added HATU (60 mg, 0.16 mmol) at room temperature. After 16 h the reaction mixture was poured into ice water, and the solid was collected by filtration. The solid was dissolved in CH₂Cl₂, and dried over Na₂SO₄. Concentration in vacuo, and purification by preparative TLC (CH₂Cl₂-MeOH, 9:1) afforded the title compound as a yellow powder. MS: 563 (M⁺−1).

### Example 46

### Preparation of 2-[[4-[4-[(4-Bromophenyl)(ethoxyimino)methyl]-1-piperidinyl]-4-methyl-1-piperidinyl]carbonyl]-3-quinoxalinol

To a solution of 4-[(4-bromophenyl)(ethoxyimino)methyl]-1-(4-methyl-4-piperidinyl)piperidine (100 mg, 0.24 mmol), 3-hydroxy-2-quinoxalinecarboxylic acid (56 mg, 0.29 mmol) and Et₃N (87 mg, 0.86 mmol) in DMF(6 mL), HATU(119 mg, 0.31 mmol) was added at room temperature and the reaction was stirred for 24 h. The reaction mixture was poured into ice water and the first crop of solid was collected by filtration. The water layer was extracted with ethyl acetate and the organic layer was washed with NaHCO₃, dried and concentrated to give the second crop of solid. The two crops of crude were combined and purified by flash chromatography (2% to 10%, MeOH/CH₂Cl₂) to afford the title compound as an orange solid. MS 579(M⁺).

### Example 47

### Preparation of 2-[[4-[4-[(4-Bromophenyl)(ethoxyimino)methyl]-1-piperidinyl]-4-methyl-1-piperidinyl]carbonyl]-1,6-naphthyridine

To a solution of 4-[(4-bromophenyl)(ethoxyimino)methyl]-1-(4-methyl-4-piperidinyl)-piperidine (50 mg, 0.1 2 mmol), 1,6-naphthyridine-2-carboxylic acid (25 mg, 0.14 mmol), Et₃N (44 mg, 0.43 mmol) in DMF (3 mL, anhydrous) was added HATU(60 mg, 0.16 mmol) at room temperature. After 16 h the reaction mixture was poured into ice water, and the solid was collected by filtration. The solid was dissolved in CH₂Cl₂, and dried over Na₂SO₄. Concentration in vacuo, and purification by preparative TLC (CH₂Cl₂-MeOH, 9:1) afforded the title compound as a light yellow powder. MS: 564 (M⁺).

### Example 48

### Preparation of 2-[[4-[4-[(4-Bromophenyl)(ethoxyimino)methyl]-1-piperidinyl]-4-methyl-1-piperidinyl]carbonyl]-1,8-naphthyridine

To a solution of 4-[(4-bromophenyl)(ethoxyimino)methyl]-1-(4-methyl-4-piperidinyl)piperidine (50 mg, 0.12 mmol), 1,8-naphthyridine-2-carboxylic acid (25 mg, 0.14 mmol), and Et₃N (44 mg, 0.43 mmol) in DMF (3 mL, anhydrous) was added HATU (60 mg, 0.16 mmol) at room temperature. After 16 h the reaction mixture was poured into ice water, and the solid was collected by filtration. The solid was dissolved in CH₂Cl₂ and dried over Na₂SO₄. Concentration in vacuo, and purification by preparative TLC (CH₂Cl₂-MeOH, 9:1) afforded the title compound as a light brown powder. MS: 564 (M⁺).

### Example 49

### Preparation of 3-[[4-[4-[(E)-(4-Bromophenyl)(ethoxyimino)methyl]-1-piperidinyl]-4-methyl-1-piperidinyl]carbonyl]-2-methyl-1,8-naphthyridine

To a solution of 4-[(E)-(4-bromophenyl)(ethoxyimino)methyl]-1-(4-methyl-4-piperidinyl)-piperidine (50 mg, 0.12 mmol), 2-methyl-1,8-naphthyridine-3-carboxylic acid (25 mg, 0.13 mmol), and Et₃N(44 mg 0.43 mmol) in DMF (3 mL) was added HATU(60 mg, 0.16 mmol) at room temperature. After 16 h the reaction mixture was poured into ice water. The solid was collected by filtration, and was re-dissolved in CH₂Cl₂; dried over Na₂SO₄. Concentration and purification by preparative TLC(CH₂Cl₂-MeOH, 9:1) afforded the title compound as a light yellow powder. ¹H NMR (CDCl₃) δ 0.94 (s, 3H), 1.20 (t, 3H), 1.24-1.88 (m, 7H), 2.00-2.17 (m, 3H), 2.42 (m, 1H), 2.74-2.84 (m, 4H), 2.94-3.10 (m, 2H), 3.35-3.54 (m, 2H), 4.06 (q, 2H), 4.19 (m, 1H), 7.11 (d, 2H), 7.47 (dd, 1H), 7.52 (d, 2H), 7.99 (s, 1H), 8.16 (d, 1H), 9.11 (m, 1H).

### Example 50

### Preparation of 3-[[4-[4-[(4-Bromophenyl)(ethoxyimino)methyl]-1-piperidinyl]-4-methyl-1-piperidinyl]carbonyl]-2-(trifluoromethyl)-1,8-naphthyridine

To a stirred solution of 4-[(4-bromophenyl)(ethoxyimino)methyl]-1-(4-methyl-4-piperidinyl)-piperidine (50 mg, 0.12 mmol), 2-trifluoromethyl-1,8-naphthyridine-3-carboxylic acid (32 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), was extracted with CH₂Cl₂ (3×10 mL). The organic phase was dried over Na₂SO₄, and concentrated in vacuo. The crude product was purified by preparative TLC to afford the title compound.

### Example 51

### Preparation of 3-[[4-[4-[(Z)-(4-Bromophenyl)(ethoxyimino)methyl]-1-piperidinyl]-4-methyl-1-piperidinyl]carbonyl]-2-methyl-1,6-naphthyridine

To a stirred solution of 4-[(Z)-(4-bromophenyl)(ethoxyimino)methyl]-1-(4-methyl-4-piperidinyl)piperidine (50 mg, 0.1 2 mmol), 2-methyl-1,6-naphthyridine-3-carboxylic acid (25 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water and filtered. The solid was dissolved in CH₂Cl₂ (2 mL) and purified by preparative TLC to afford title compound as a light yellow solid. ¹H NMR (CDCl₃, 400 MHz) δ 0.93 (s, 3H), 1.2 (t, 3H), 1.24-1.96 (m, 7H), 1.98-2.2 (m, 3H), 2.36-2.5 (m, 1H), 2.72-2.9 (m, 4H), 2.92-3.14 (m, 2H), 3.38-3.58 (m, 2H), 4.04 (q, 2H), 4.15-4.3 (m,1H), 7.08-7.14 (m, 2H), 7.48-7.56 (m, 2H), 7.8-7.88 (m, 1H), 8.07 (s, 1H), 8.72-8.78 (d, 1H), 9.18-9.24 (s, 1H).

### Example 52

### Preparation of 2-[[4-[4-[(4-Bromophenyl)(ethoxyimino)methyl]-1-piperidinyl]-4-methyl-1-piperidinyl]carbonyl]-1H-indole

To a stirred solution of 4-[(4-bromophenyl)(ethoxyimino)methyl]-1-(4-methyl-4-piperidinyl)piperidine (50 mg, 0.12 mmol), 1H-indole-2-carboxylic acid (21 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL) and extracted with CH₂Cl₂ (3×10 mL). The organic phase was dried over Na₂SO₄, and concentrated in vacuo. The crude product was purified by HPLC to afford the title compound. MS: 552 (M⁺+1).

### Example 53

### Preparation of 3-[[4-[4-[(4-Bromophenyl)(ethoxyimino)methyl]-1-piperidinyl]-4-methyl-1-piperidinyl]carbonyl]-1-methyl-1H-indole

To a stirred solution of 4-[(4-bromophenyl)(ethoxyimino)methyl]-1-(4-methyl-4-piperidinyl)piperidine (50 mg, 0.12 mmol), 1-methyl-1H-indole-3-carboxylic acid (27 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), was extracted with CH₂Cl₂ (3×10 mL). The organic phase was dried over Na₂SO₄, and concentrated in vacuo. The crude product was purified by preparative TLC to afford the title compound. MS: 565 (M⁺).

### Example 54

### Preparation of 3-[[4-[4-[(4-Bromophenyl)(ethoxyimino)methyl]-1-piperidinyl]-4-methyl-1-piperidinyl]carbonyl]-1H-indole

To a stirred solution of 4-[(4-bromophenyl)(ethoxyimino)methyl]-1-(4-methyl-4-piperidinyl)piperidine (50 mg, 0.12 mmol), 1H-indole-3-carboxylic acid (21 mg, 0.13 mmol), and Et₃N (24 mg, 0.24 mmol) in DMF (2 mL), HATU (61 mg, 0.16 mmol) was added at room temperature. After 16 h the mixture was poured into ice water (10 mL) and extracted with CH₂Cl₂ (3×10 mL). The organic phase was dried over Na₂SO₄, and concentrated in vacuo. The crude product was purified by preparative TLC to afford the title compound.

### Example 55

### Preparation of 5-[[4-[4-[(4-Bromophenyl)(ethoxyimino)methyl]-1-piperidinyl]-4-methyl-1-piperidinyl]carbonyl]-1H-indole

To a stirred solution of 4-[(4-bromophenyl)(ethoxyimino)methyl]-1-(4-methyl-4-piperidinyl)piperidine (50 mg, 0.12 mmol), 1H-indole-5-carboxylic acid (21 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and was extracted with CH₂Cl₂ (3×10 mL). The organic phase was dried over Na₂SO₄, and concentrated in vacuo. The crude product was purified by preparative TLC to afford the title compound. MS: 550 (M⁺−1).

### Example 56

### Preparation of 5-[[4-[4-[(Z)-(4-Bromophenyl)(ethoxyimino)methyl]-1-piperidinyl]-4-methyl-1-piperidinyl]carbonyl]-1-methyl-1H-indole

To a stirred solution of 4-[(Z)-(4-bromophenyl)(ethoxyimino)methyl]-1-(4-methyl-4-piperidinyl)piperidine (50 mg, 0.12 mmol), 1-methyl-1H-indole-5-carboxylic acid (23 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and solid was collected by filtration. The solid was dissolved in CH₂Cl₂, and purified by preparative TLC to afford the title compound. ¹H NMR (CDCl₃, 400 MHz) δ 0.93 (s, 3H), 1.3 (t, 3H), 1.34-2.0 (m, 8H), 2.08-2.3 (m, 2H), 2.7-3.1 (m, 2H), 3.1-3.4 (m, 2H), 3.4-3.68 (m, 2H), 3.8 (s, 3H), 3.86-4.1 (m, 1H), 4.12-4.24 (q, 2H), 6.48-6.54 (m, 1H), 7.08-7.14 (d, 1H), 7.2-7.34 (m, 4H), 7.44-7.54 (m, 2H), 7.66-7.74 (m, 1H).

### Example 57

### Preparation of 5-[[4-[4-[(Z)-(4-Bromophenyl)(ethoxyimino)methyl]-1-piperidinyl]-4-methyl-1-piperidinyl]carbonyl]-1-ethyl-1H-indole

To a stirred solution of 4-[(Z)-(4-bromophenyl)(ethoxyimino)methyl]-1-(4-methyl-4-piperidinyl)piperidine (50 mg, 0.12 mmol), 1-ethyl-1H-indole-5-carboxylic acid (25 mg, 0.13 mmol), and Et₃N (22.3 mg, 0.22 mmol) in DMF (2 mL), HATU (61 mg, 0.16 mmol) was added at room temperature. After 16 h the mixture was poured into ice water (10 mL) and extracted with CH₂Cl₂ (3×10 mL). The organic phase was dried over Na₂SO₄, and concentrated in vacuo. The crude product was purified by preparative TLC to afford the title compound. ¹H NMR (CDCl₃, 400 MHz) δ 0.93 (s, 3H), 1.3(t, 3H), 1.3-2.26 (m, 14H), 2.28-2.5 (m, 1H), 2.6-3.2 (m, 2H), 3.22-4.0 (m, 3H), 4.0-4.1 (q, 2H), 4.12-4.24 (q, 2H), 6.48-6.58 (m, 1H), 7.08-7.14 (m, 2H), 7.14-7.18 (m, 1H), 7.23-7.3 (m, 1H), 7.3-7.38 (m, 1H), 7.48-7.56 (m, 2H), 7.66-7.72 (m, 1H).

### Example 58

### Preparation of 2-[[4-[4-[(E)-(4-Bromophenyl)(ethoxyimino)methyl]-1-piperidinyl]-4-methyl-1-piperidinyl]carbonyl]-1-methyl-1H-indole

To a stirred solution of 4-[(4-bromophenyl)(ethoxyimino)methyl]-1-(4-methyl-4-piperidinyl)piperidine (50 mg, 0.12 mmol), 1-methyl-1H-indole-2-carboxylic acid (22 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and solid was collected by filtration. The solid was dissolved in CH₂Cl₂, and purified by preparative TLC to afford the title compound. ¹H NMR (CDCl₃, 400 MHz) δ 0.93 (s, 3H), 1.3 (t, 3H), 1.34-2.08 (m, 8H),2.1-2.3 (m, 2H), 2.7-3.1 (m, 2H),3.1-3.3 (m, 1H),3.44-3.68 (m, 3H),3.82 (s, 3H),3.89-4.1 (m, 1H), 4.14-4.24 (q, 2H),6.58-6.64 (s, 1H) 7.08-7.18 (m, 1H), 7.2-7.32 (m, 4H), 7.46-7.54 (d, 2H), 7.56-7.66 (m, 1H).

### Example 59

### Preparation of 2-[[4-[4-[(4-Bromophenyl)(ethoxyimino)methyl]-1-piperidinyl]-4-methyl-1-piperidinyl]carbonyl]-1-ethyl-1H-indole

To a stirred solution of 4-[(4-bromophenyl)(ethoxyimino)methyl]-1-(4-methyl-4-piperidinyl)-piperidine (50 mg, 0.12 mmol), 1-ethyl-1H-indole-2-carboxylic acid (25 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and was extracted with CH₂Cl₂ (3×10 mL). The organic phase was dried over Na₂SO₄, and concentrated in vacuo. The crude product was purified by preparative TLC to afford the title compound.

### Example 60

### Preparation of 3-[[4-[4-[(Z)-(4-Bromophenyl)(ethoxyimino)methyl]-1-piperidinyl]-4-methyl-1-piperidinyl]carbonyl]-1-ethyl-1H-indole

To a stirred solution of 4-[(Z)-(4-bromophenyl)(ethoxyimino)methyl]-1-(4-methyl-4-piperidinyl)-piperidine (50 mg, 0.12 mmol), 1-ethyl-1H-indole-3-carboxylic acid (25 mg, 0.16 mmol), and Et₃N (24.3 mg, 0.24 mmol) in DMF (2 mL), HATU (60.8 mg, 0.16 mmol) was added at room temperature. After 16 h the mixture was poured into ice water and filtered. The solid was dissolved in CH₃CN (2 mL) and purified by HPLC to afford title compound, trifluoroacetic acid salt, as a white solid. ¹H NMR (CDCl₃, 400 MHz) δ 1.16-1.36 (m, 4H), 1.42-1.56 (m, 6H), 1.8-1.92 (m, 2H), 1.96-2.5 (m, 6H), 2.54-2.84 (m, 1H), 2.96-2.3.3 (m, 3H), 3.44-3.56 (m, 1H), 3.68-3.80 (m, 1H), 4.0-4.26 (m, 4H), 4.4-4.6 (m, 2H), 7.08-7.3 (m, 5H), 7.32-7.4 (m, 1H), 7.42-7.66 (m, 3H).

### Example 61

### Preparation of 4-[[4-[4-[(Z)-(4-Bromophenyl)(ethoxyimino)methyl]-1-piperidinyl]-4-methyl-1-piperidinyl]carbonyl]-1-methyl-1H-indole

To a stirred solution of 4-[(Z)-(4-bromophenyl)(ethoxyimino)methyl]-1-(4-methyl-4-piperidinyl)piperidine (50 mg, 0.12 mmol), 1-methyl-1H-indole-4-carboxylic acid (23 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and extracted with CH₂Cl₂ (3×10 mL). The organic phase was dried over Na₂SO₄, and concentrated in vacuo. Purification by preparative TLC afforded title compound as a light yellow oil. ¹H NMR (CDCl₃, 400 MHz) δ 0.93 (s, 3H), 1.2 (t, 3H), 1.24-2.2 (m, 11H), 2.34-2.46 (m, 1H), 2.76-2.9 (m, 1H), 2.9-3.1 (m, 1H), 3.1-3.3 (m, 1H), 3.3-3.7 (m, 2H), 3.78-3.84 (s, 3H), 4.02-4.18 (q, 2H), 6.4-6.6 (m, 1H), 7.08-7.14 (m, 4H), 7.19-7.25 (m, 1H), 7.32-7.36 (m, 1H), 7.48-7.56 (m, 2H).

### Example 62

### Preparation of 6-[[4-[4-[(Z)-(4-Bromophenyl)(ethoxyimino)methyl]-1-piperidinyl]-4-methyl-1-piperidinyl]carbonyl]-1-methyl-1H-indole

To a stirred solution of 4-[(Z)-(4-bromophenyl)(ethoxyimino)methyl]-1-(4-methyl-4-piperidinyl)piperidine (50 mg, 0.12 mmol), 1-methyl-1H-indole-6-carboxylic acid (23 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and extracted with CH₂Cl₂ (3×10 mL). The organic phase was dried over Na₂SO₄, and concentrated in vacuo. Purification by preparative TLC afforded title compound as a light yellow oil. ¹H NMR (CDCl3, 400 MHz) δ 0.93 (s, 3H), 1.2 (t, 3H), 1.24-2.0 (m, 9H), 2.0-2.3 (m, 2H), 2.3-2.5 (m, 1H), 2.7-3.2 (m, 2H), 3.3-3.7 (m, 3H), 3.78-3.84 (s, 3H), 4.02-4.2 (q, 2H), 6.4-6.6 (m, 1H), 7.08-7.14 (m, 4H), 7.44-7.48 (s, 1H), 7.48-7.54 (m, 2H), 7.56-7.62 (m, 1H).

### Example 63

### Preparation of 4-[[4-[4-[(Z)-(4-Bromophenyl)(ethoxyimino)methyl]-1-piperidinyl]-4-methyl-1-piperidinyl]carbonyl]-1-ethyl-1H-indole

To a stirred solution of 4-[(Z)-(4-bromophenyl)(ethoxyimino)methyl]-1-(4-methyl-4-piperidinyl)piperidine (50 mg, 0.12 mmol), 1-ethyl-1H-indole-4-carboxylic acid (25 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and extracted with CH₂Cl₂ (3×10 mL). The organic phase was dried over Na₂SO₄, and concentrated in vacuo. Purification by preparative TLC afforded title compound as a light yellow oil. ¹H NMR (CDCl₃, 400 MHz) δ 0.93 (s, 3H), 1.2 (t, 3H), 1.24-1.86 (m, 11H), 1.86-2.3 (m, 3H), 2.3-2.5 (m, 1H), 2.78-2.9 (m,1H),2.92-3.1 (m,1H), 3.1-3.3 (m,1H), 3.3-3.7 (m, 2H), 4.02-4.14 (q, 2H), 4.14-4.24 (q, 2H), 6.4-6.6 (m, 1H), 7.08-7.14 (m, 3H), 7.15-7.25 (m, 2H), 7.34-7.4 (m, 1H), 7.48-7.58 (m, 2H).

### Example 64

### Preparation of 6-[[4-[4-[(Z)-(4-Bromophenyl)(ethoxyimino)methyl]-1-piperidinyl]-4-methyl-1-piperidinyl]carbonyl]-1-ethyl-1H-indole

To a stirred solution of 4-[(Z)-(4-bromophenyl)(ethoxyimino)methyl]-1-(4-methyl-4-piperidinyl)piperidine (50 mg, 0.12 mmol), 1-methyl-1H-indole-6-carboxylic acid (25 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and extracted with CH₂Cl₂ (3×10 mL). The organic phase was dried over Na₂SO₄, and concentrated in vacuo. Purification by preparative TLC afforded title compound as a brown oil. ¹H NMR (CDCl₃, 400 MHz) δ 0.93 (s, 3H), 1.2 (t, 3H), 1.24-2.0 (m, 12H), 2.04-2.24 (m, 2H), 2.34-2.5 (m, 1H), 2.78-3.3 (m, 2H), 3.3-4.02 (m, 3H), 4.02-4.14 (q, 2H), 4.14-4.26 (q, 2H), 6.4-6.6 (m,1H), 7.08-7.16 (m, 3H), 7.16-7.23 (m, 1H), 7.46-7.49 (s, 1H), 7.49-7.56 (m, 2H), 7.57-7.62 (m, 1H).

### Example 65

### Preparation of 6-[[4-[4-[(Z)-(4-Bromophenyl)(ethoxyimino)methyl]-1-piperidinyl]-4-methyl-1-piperidinyl]carbonyl]-1H-indole

To a stirred solution of 4-[(Z)-(4-bromophenyl)(ethoxyimino)methyl]-1-(4-methyl-4-piperidinyl)piperidine (50 mg, 0.12 mmol), 1H-indole-6-carboxylic acid (21 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and extracted with CH₂Cl₂ (3×10 mL). The organic phase was dried over Na₂SO₄, and concentrated in vacuo. Purification by preparative HPLC afforded title compound, trifluoroacetic acid salt as a white solid ¹H-NMR (CDCl₃, 400 MHz) δ1.2 (m, 3H), 1.4 (dd, 3H), 1.7-1.9 (m, 2H), 1.9-2.5 (m, 5H), 2.5-3.0 (m, 3H), 3.0-3.3 (m, 1H), 3.3-3.95 (m, 5H), 4.0-4.16 (m, 2H), 4.18-4.6 (m, 1H), 6.4-6.6 (m, 1H), 7.09-7.22 (m, 3H), 7.29-7.34 (m, 1H), 7.48-7.58 (m, 3H), 7.59-7.68 (m, 1H), 9.3(m, 1H).

### Example 66

### Preparation of 4-[[4-[4-[(Z)-(4-Bromophenyl)(ethoxyimino)methyl]-1-piperidinyl]-4-methyl-1-piperidinyl]carbonyl]-1-methyl-1H-indole

To a stirred solution of 4-[(Z)-(4-bromophenyl)(ethoxyimino)methyl]-1-(4-methyl-4-piperidinyl)piperidine (50 mg, 0.12 mmol), 1-methyl-1H-indole-4-carboxylic acid (21 mg, 0.13 mmol), and Et₃N (24.3 mg, 0.24 mmol) in DMF (2 mL), HATU (60.8 mg, 0.16 mmol) was added at room temperature. After 16 h the mixture was poured into ice water (10 mL), and was extracted with CH₂Cl₂ (3×10 mL). The organic phase was dried over Na₂SO₄, and concentrated in vacuo. Purification by preparative TLC afforded title compound as a green oil. ¹H NMR (CDCl₃, 400 MHz) δ 0.93 (s, 3H), 1.2 (t, 3H), 1.24-2.3 (m, 11H), 2.3-2.5 (m, 1H), 2.7-3.3 (m, 3H), 3.32-3.7 (m, 2H), 4.02-4.18 (q, 2H), 6.4-6.6 (m, 1H), 7.08-7.24 (m, 4H), 7.36-7.42 (m, 1H), 7.48-7.56 (s, 2H), 8.3-8.6 (m, 1H).

### Example 67

### Preparation of 1-[1-(Benzo[b]thien-3-ylcarbonyl)-4-methyl-4-piperidinyl]-4-(4-bromophenyl)(ethoxyimino)methyl]piperidine

To a stirred solution of 4-[(Z)-(4-bromophenyl)(ethoxyimino)methyl]-1-(4-methyl-4-piperidinyl)piperidine (50 mg, 0.12 mmol), benzo[b]thiophene-3-carboxylic acid (22 mg, 0.13 mmol), and Et₃N (24.3 mg, 0.24 mmol) in DMF (2 mL), HATU (61 mg, 0.16 mmol) was added at room temperature. After 16 h the mixture was poured into ice water (10 mL), and was extracted with CH₂Cl₂ (3×10 mL). The organic phase was dried over Na₂SO₄, and concentrated in vacuo. Purification by preparative TLC afforded title compound as a white solid. MS: 569 (M⁺+1).

### Example 68

### Preparation of 4-[[4-[4-[(4-bromophenyl)hydroxymethyl]-4-piperidin-1-yl]-4-methylpiperidin-1-yl]carbonyl]quinoline

To a stirred solution of 4-[4-(4-bromophenyl)hydroxymethyl]-1-piperidinyl]-4-methyl-1-piperidinecarboxylic acid 1,1-diemthylethyl ester (1.03 g, 2.2 mmol) in CH₂Cl₂ (10 mL) was added TFA at room temperature. After 2 h the reaction mixture was concentrated in vacuo, and dried under vacuum. The product was dissolved in DMF (10 mL), and quinoline-4-carboxylic acid (450 mg, 2.6 mmol), Et₃N (1.0 mL, 7.2 mmol), and HATU (1.1 g, 2.9 mmol) was added at room temperature. After 16 h, the reaction mixture was poured into ice water while stirring vigorously. The solid was collected by filtration, and was re-dissolved in CH₂Cl₂ and dried over Na₂SO₄. Concentration and purification by flash chromatography (CH₂Cl₂-MeOH, 100:1 to 100:2 to 100:4) afforded the title compound as a brown powder. MS: 523.1 (M⁺+1). ¹H NMR (CDCl₃) δ 0.92 (s, 3H), 1.27-2.14 (m, 10H), 2.72-3.00 (m, 3H), 3.33 (m, 1H), 3.60 (m, 1H), 4.21 (m, 1H), 4.38 (m, 1H), 7.19 (m, 2H), 7.31 (m, 1H), 7.48 (m, 2H), 7.62 (m, 1H), 7.77-7.86 (m, 2H), 8.15 (br.d, 1H), 8.94 (m, 1H).

### Example 69

### Preparation of 4-[[4-[4-[(4-Bromophenyl)(2-pyridinyloxy)methyl]-1-piperidinyl]-4-methyl-1-piperidinyl]carbonyl]-7-chloroquinoline

To a solution of 4-[(4-bromophenyl)(2-pyridinyloxy)methyl]-1-(4-methyl-4-piperidinyl)piperidine (80 mg, 0.18 mmol), 7-chloro-quinoline-4-carboxylic acid (45 mg, 0.22 mmol), Et₃N (31 mg, 0.3 mmol) in DMF (5 mL) was added HATU (104 mg, 0.27 mmol) at room temperature. After 16 h the reaction mixture was poured into ice water. The solid was collected by filtration, and was re-dissolved in CH₂Cl₂ and dried over Na₂SO₄. Concentration and purification by flash chromatography CH₂Cl₂-MeOH, 95:5 to 9:1) afforded title compound as a light yellow powder. ¹H NMR (CDCl₃) δ 0.91 (s, 3H), 1.10-2.15 (m, 11H), 2.74 (m, 1H), 2.95 (m, 2H), 3.32 (m, 1H), 3.52 (m, 1H), 4.24 (m, 1H), 5.80 (m, 1H), 6.75 (m, 2H), 7.25 (m, 1H), 7.30 (d, 1H), 7.42 (m, 2H), 7.49-7.60 (m, 2H), 7.78 (t, 1H), 8.04 (m, 1H), 8.14 (d, 1H), 8.95 (d, 1H).

### Example 70

### Preparation of 4-[[4-[4-[(4-Bromophenyl)(2-pyridinyloxy)methyl]-1-piperidinyl]-4-methyl-1-piperidinyl]carbonyl]quinoline

To a solution of 4-[(4-bromophenyl)(2-pyridinyloxy)methyl]-1-(4-methyl-4-piperidinyl)piperidine (220 mg, 0.5 mmol), quinoline-4-carboxylic acid (110 mg, 0.64 mmol), and Et₃N (192 mg, 1.9 mmol) in DMF (5 mL) was added HATU (260 mg, 0.68 mmol) at room temperature. After 16 h the reaction mixture was poured into ice water. The solid was collected by filtration, dissolved in CH₂Cl₂, and dried over Na₂SO₄. Concentration and purification by flash chromatography (CH₂Cl₂-MeOH, 95:5 to 9:1) afforded the title compound as a light yellow powder. ¹H NMR(CDCl₃, 400 MHz): LC-MS. 598 (M⁺). ¹H NMR (CDCl₃) δ 0.91 (s, 3H), 1.16-2.14 (m, 11H), 2.74 (m, 1H), 2.96 (m, 2H), 3.30 (m, 1H), 3.56 (m, 1H), 4.06 (q, 2H), 4.24 (m, 1H), 5.80 (m, 1H), 6.75 (m, 2H), 7.25 (m, 1H), 7.30 (d, 1H), 7.42 (m, 2H), 7.50-7.65 (m, 2H), 7.75 (m, 1H), 7.85 (m, 1H), 8.05 (m, 1H), 8.15 (d, 1H), 8.95 (d, 1H).

### Example 71

### Preparation of 4-[[4-[4-[(4-Bromophenyl)(2-pyridinyloxy)methyl]-1-piperidinyl]-4-methyl-1-piperidinyl]carbonyl]-1-hydroxyquinolinium

To a stirred solution of 4-[(4-bromophenyl)(2-pyridinyloxy)methyl]-1-(4-methyl-4-piperidinyl)piperidine (50 mg, 0.11 mmol), 4-carboxy-1-hydroxyquinolinium (23 mg, 0.12 mmol), and Et₃N (22.4 mg, 0.22 mmol) in DMF (2 mL), HATU (55 mg, 0.14 mmol) was added at room temperature. After 16 h the mixture was poured into ice water (10 mL), and extracted with CH₂Cl₂ (3×10 mL). The organic phase was dried over Na₂SO₄, and concentrated in vacuo. The crude product was purified by preparative TLC to afford the title compound. MS: 616 (M⁺−1). ¹H NMR (CDCl₃, 400 MHz) δ 0.93 (s, 3H), 1.0-1.58 (m, 5H), 1.58-2.2 (m, 7H), 2.6-3.1 (m, 3H), 3.1-3.65 (m, 2H), 4.1-4.3 (m, 1H), 5.7-5.95 (m, 1H), 6.5-6.86 (m, 2H), 7.22 (m, 2H), 7.34-7.6 (m, 3H), 7.6-7.94 (m, 3H), 7.95-8.1 (m, 1H), 8.42-8.55 (d, 1H), 8.66-8.84 (d, 1H).

### Example 72

### Preparation of 5-[[4-[4-[(4-Bromophenyl)(2-pyridinyloxy)methyl]-1-piperidinyl]-4-methyl-1-piperidinyl]carbonyl]quinoline

To a stirred solution of 4-[(4-bromophenyl)(2-pyridinyloxy)methyl]-1-(4-methyl-4-piperidinyl)piperidine (50 mg, 0.11 mmol), 5-quinolinecarboxylic acid (21 mg, 0.12 mmol), and Et₃N (22 mg, 0.22 mmol) in DMF (2 mL), HATU (55 mg, 0.14 mmol) was added at room temperature. After 16 h the mixture was poured into ice water (10 mL), and extracted with CH₂Cl₂ (3×10 mL). The organic phase was dried over Na₂SO₄, and concentrated in vacuo. The crude product was purified by preparative TLC to afford the title compound. MS: 599 (M⁺). ¹H NMR (CDCl₃, 400 MHz) δ 0.93 (d, 3H), 1.0-1.58 (m, 5H), 1.58-2.2 (m, 7H), 2.5-3.1 (m, 3H), 3.1-3.65 (m, 2H), 4.1-4.4 (m, 1H), 5.7-5.95 (m, 1H), 6.5-6.9 (m, 2H), 7.22 (m, 2H), 7.3-7.6 (m, 4H), 7.6-7.8 (m, 1H), 7.95-8.3 (m, 3H), 8.7-8.95 (m, 1H).

### Example 73

### Preparation of 5-[[4-[4-[(4-Bromophenyl)(2-pyridinyloxy)methyl]-1-piperidinyl]-4-methyl-1-piperidinyl]carbonyl]-1-hydroxyquinolinium

To a stirred solution of 4-[(4-bromophenyl)(2-pyridinyloxy)methyl]-1-(4-methyl-4-piperidinyl)piperidine (50 mg, 0.11 mmol), 5-carboxy-1-hydroxy-quinolinium (23 mg, 0.13 mmol), and Et₃N (22 mg, 0.22 mmol) in DMF (2 mL), HATU (55 mg, 0.14 mmol) was added at room temperature. After 16 h the mixture was poured into ice water (10 mL), and extracted with CH₂Cl₂ (3×10 mL). The organic phase was dried over Na₂SO₄, and concentrated in vacuo. The crude product was purified by preparative TLC to afford the title compound. MS: 616 (M⁺−1). ¹H NMR (CDCl₃, 400 MHz) δ 0.93 (d, 3H), 1.0-1.6 (m, 5H), 1.6-2.4 (m, 7H), 2.5-3.1 (m, 3H), 3.1-3.65 (m, 2H), 4.0-4.4 (m, 1H), 5.78 (m, 1H), 6.5-6.9 (m, 1H), 7.22 (m, 2H), 7.24-7.44 (m, 2H), 7.45-7.64 (m, 2H), 7.66-7.84 (m, 2H), 7.96-8.1 (m, 1H), 8.44-8.64 (m, 1H), 8.7-8.84 (d, 1H).

### Example 74

### Preparation of 4-[[4-[4-[1-(4-Bromophenyl)-2,2,2-trifluoro-1-[(trimethylsilyl)oxy]ethyl]-1-piperidinyl]-4-methyl-1-piperidinyl]carbonyl]-7-chloro-quinoline & 4-[[4-[4-[1-(4-bromophenyl)-(2,2,2-trifluoro-1-hydroxy)ethyl]-1-piperidinyl]-4-methyl-1-piperidinyl]carbonyl]-7-chloroquinoline

To a solution of 4-[[4-[4-(4-bromobenzoyl)-1-piperidinyl]-4-methyl-1-piperidinyl]carbonyl]-7-chloroquinoline (40 mg, 0.07 mmol) in CH₂Cl₂ (2 mL) was added TMSCF₃ (0.1 mL, 0.68 mmol) and TMAF.4H₂O (cat.) at room temperature under N₂. After 2 h, a solution of TFA-H₂O (1:1) was added, and the reaction mixture was stirred for an additional 2 h. After removal of solvents, the residue was purified by preparative TLC (CH₂Cl₂-MeOH, 9:1) to afford a light brown syrup. 4-[[4-[4-[1-(4-Bromophenyl)-2,2,2-trifluoro-1-[(trimethylsilyl)oxy]ethyl]-1-piperidinyl]-4-methyl-1-piperidinyl]carbonyl]-7-chloro-quinoline: ¹H NMR (CDCl₃) δ 0.23 (s, 9H), 0.91 (s, 3H), 1.08-2.12 (m, 11H), 2.60-3.40 (m, 3H), 3.26 (m, 1H), 3.51 (m, 1H), 4.22 (m, 1H), 7.28-7.36 (m, 3H), 7.46-7.58 (m, 3H), 7.76 (br. dd, 1H), 8.14 (m, 1H), 8.94 (m, 1H).

4-[[4-[4-[1-(4-bromophenyl)-(2,2,2trifluoro-1-hydroxy)ethyl]-1-piperidinyl]-4-methyl-1-piperidinyl]carbonyl]-7-chloroquinoline ¹H NMR (CDCl₃): δ 0.94 (br.s, 3H), 1.10-2.26 (m, 11H), 2.56-3.10 (m, 3H), 3.30 (m, 1H), 3.60 (m, 1H), 4.16 (m, 1H), 7.30 (m, 1H), 7.42 (m, 2H), 7.47-7.62 (m, 3H), 7.78 (d, 1H), 8.15 (m, 1H), 8.70 (m, 1H).

### Example 75

### Preparation of 4-[[4-[4-[1-(4-Bromophenyl)ethenyl]-1-piperidinyl]-4-methyl-1-piperidinyl]carbonyl]quinoline

To a stirred solution of 4-[4-[1-(4-bromophenyl)ethenyl]-4-piperidinyl]4-methyl-1-piperidinecarboxylic acid, dimethylethyl ester (51 mg, 0.11 mmol) in CH₂Cl₂ (2 mL) was added TFA (1 mL) at room temperature. After 2 h, the reaction was concentrated in vacuo and dried over vacuum. The crude product was dissolved in DMF (2 mL), then 4-quinolinecarboxylic acid (21 mg, 0.12 mmol), Et₃N (22 mg, 0.22 mmol), and HATU (55 mg, 0.14 mmol) was added at room temperature. After 16 h the mixture was poured into ice water (10 mL), and extracted with CH₂Cl₂ (3×10 mL). The organic phase was dried over Na₂SO₄, and concentrated in vacuo. The crude product was purified by preparative TLC to afford the title compound. MS: 518 (M⁺). ¹H NMR (CDCl₃, 400 MHz) δ 0.93 (d, 3H), 1.1-1.62 (m, 4H), 1.62-1.9 (m, 3H), 1.98-2.22 (m, 3H), 2.24-2.42 (m, 1H), 2.7-2.88 (m, 1H), 2.9-3.1 (m, 1H), 3.24-3.42 (m, 1H), 3.5-3.7 (m, 1H), 4.17-4.4 (m, 1H), 5.0-5.1 (s, 1H), 5.1-5.22 (s, 1H), 7.14-7.23 (m, 2H), 7.28-7.36 (m, 1H), 7.4-7.48 (m, 2H), 7.54-7.66 (m, 1H), 7.72-7.9 (m, 2H), 8.1-8.2 (d, 1H), 8.9-9.0 (m, 1H).

### Example 76

### Preparation of 1-Methyl-4-[[4-methyl-4-[(3S)-3-methyl-4-[(1R)-1-[4-(trifluoromethyl)phenyl]ethyl]piperazin-1-yl]-1-piperidinyl]carbonyl]-1H-indole

To a solution of 4-methyl-4-[(3S)-3-methyl-4-[(1R)-1-[4-(trifluoromethyl)phenyl]ethyl]piperazin-1-yl]-1-piperidinecarboxylic acid-dimethylethyl ester (127 mg, 0.27 mmol) in CH₂Cl₂ (3 mL) was added trifluoromethyl acetic acid (1.5 mL) at room temperature. After 2 h the reaction mixture was concentrated in vacuo, and dried under vacuum for 3 h. Re-dissolved the product in DMF (5 mL), then 1-methyl-1H-indole-4-carboxylic acid (52 mg, 0.30 mmol), Et₃N (55 mg, 0.54 mmol), and HATU (134 mg, 0.35 mmol) was added successively at room temperature. After 16 h the reaction mixture was poured into ice water (10 mL), and the mixture was extracted with EtOAc (3×20 mL). The organic phase was washed with NaHCO₃ (10 mL, sat.) and brine (10 mL), and dried over Na₂SO₄. Concentration in vacuo, and purification by column chromatography (CH₂Cl₂-MeOH, 9:1) afforded the title compound as a yellow solid. MS: 526 (M⁺). ¹H NMR (CDCl₃, 400 MHz):δ: 0.9(s, 3H), 1.14 (d, 3H), 1.3 (d, 4H), 1.4-1.8 (m, 2H), 1.82-2.05 (m, 1H), 2.18-2.8 (m, 6H), 2.9-3.1 (m, 1H), 3.1-3.3 (m, 1H), 3.3-3.7 (m, 2H), 3.8 (s, 3H), 3.9-4.3 (m, 2H), 6.48 (m, 1H), 7.05-7.16 (m, 2H), 7.18-7.28 (m, 1H), 7.3-7.38 (m, 1H), 7.46-7.6 (m, 4H).

### Example 77

This Example illustrates the preparation of representative pharmaceutical compositions for oral administration containing a compound of the invention, or a pharmaceutically acceptable salt thereof:

| A. Ingredients | % wt./wt. |
|---|---|
| Compound of the invention | 20.0% |
| Lactose | 79.5% |
| Magnesium stearate | 0.5% |

The above ingredients are mixed and dispensed into hard-shell gelatin capsules containing 100 mg each, one capsule would approximate a total daily dosage.

| B. Ingredients | % wt./wt. |
|---|---|
| Compound of the invention | 20.0% |
| Magnesium stearate | 0.9% |
| Starch | 8.6% |
| Lactose | 69.6% |
| PVP (polyvinylpyrrolidine) | 0.9% |

The above ingredients with the exception of the magnesium stearate are combined and granulated using water as a granulating liquid. The formulation is then dried, mixed with the magnesium stearate and formed into tablets with an appropriate tableting machine.

| C. Ingredients | | |
|---|---|---|
| Compound of the invention | 0.1 | g |
| Propylene glycol | 20.0 | g |
| Polyethylene glycol 400 | 20.0 | g |
| Polysorbate 80 | 1.0 | g |
| Water | q.s. 100 | mL |

The compound of the invention is dissolved in propylene glycol, polyethylene glycol 400 and polysorbate 80. A sufficient quantity of water is then added with stirring to provide 100 mL of the solution, which is filtered and bottled.

| D. Ingredients | % wt./wt. |
|---|---|
| Compound of the invention | 20.0% |
| Peanut Oil | 78.0% |
| Span 60 | 2.0% |

The above ingredients are melted, mixed and filled into soft elastic capsules.

| E. Ingredients | % wt./wt. |
|---|---|
| Compound of the invention | 1.0% |
| Methyl or carboxymethyl cellulose | 2.0% |
| 0.9% saline | q.s. 100 mL |

The compound of the invention is dissolved in the cellulose/saline solution, filtered and bottled for use.

### Example 78

This Example illustrates the preparation of a representative pharmaceutical formulation for parenteral administration containing a compound of the invention, or a pharmaceutically acceptable salt thereof:

| Ingredients | | |
|---|---|---|
| Compound of the invention | 0.02 | g |
| Propylene glycol | 20.0 | g |
| Polyethylene glycol 400 | 20.0 | g |
| Polysorbate 80 | 1.0 | g |
| 0.9% Saline solution | q.s. 100 | mL |

The compound of the invention is dissolved in propylene glycol, polyethylene glycol 400 and polysorbate 80. A sufficient quantity of 0.9% saline solution is then added with stirring to provide 100 mL of the I.V. solution, which is filtered through a 0.2 m membrane filter and packaged under sterile conditions.

### Example 79

This Example illustrates the preparation of a representative pharmaceutical composition in suppository form containing a compound of the invention, or a pharmaceutically acceptable salt thereof:

| Ingredients | % wt./wt. |
|---|---|
| Compound of the invention | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

### Example 80

This Example illustrates the preparation of a representative pharmaceutical formulation for insufflation containing a compound of the invention, or a pharmaceutically acceptable salt thereof:

| Ingredients | % wt./wt. |
|---|---|
| Micronized compound of the invention | 1.0% |
| Micronized lactose | 99.0% |

The ingredients are milled, mixed, and packaged in an insufflator equipped with a dosing pump.

### Example 81

This Example illustrates the preparation of a representative pharmaceutical formulation in nebulized form containing a compound of the invention, or a pharmaceutically acceptable salt thereof:

| Ingredients | % wt./wt. |
|---|---|
| Compound of the invention | 0.005% |
| Water | 89.995% |
| Ethanol | 10.000% |

The compound of the invention is dissolved in ethanol and blended with water. The formulation is then packaged in a nebulizer equipped with a dosing pump.

### Example 82

This Example illustrates the preparation of a representative pharmaceutical formulation in aerosol form containing a compound of the invention, or a pharmaceutically acceptable salt thereof:

| Ingredients | % wt./wt. |
|---|---|
| Compound of the invention | 0.10% |
| Propellant 11/12 | 98.90% |
| Oleic acid | 1.00% |

The compound of the invention is dispersed in oleic acid and the propellants. The resulting mixture is then poured into an aerosol container fitted with a metering valve.

### Example 83

CCR-5 Receptor MIP-1a Scintillation Proximity Binding Assay

A) Assay Buffer: 50 mM Hepes, 5 mM MgCl2, 1 mM CaCl₂, 30 ug/ml bacitracin, 0.1% BSA, pH 7.4.

B) Ligand: MIP-la labeled with I-125 at 20,000-25,000 cpm/well. Non specific binding (nsb) was defined as bound cpm in the presence of 100 nM unlabeled MIP-1β.

C) Cells: Human embryonic kidney, (HEK-293) expressing human CCR-5 and CD4 pretreated overnight with 5 mM sodium butyrate. Harvest cells with calcium and magnesium free phosphate buffered saline. Cell number is counted with hemacytometer. Cell number per assay point was selected so the total counts per minute (cpm) bound was approximately 10% of the total cpms I-125-MIP-1a added per assay point.

D) Beads: Use wheatgerm agglutinin coated scintillation proximity assay beads (sold by Amersham Pharmacia Biotech Inc.) hydrated with the assay buffer for at least an hour before use. Final bead concentration was 0.2 mg beads per well.

E) Scintillation Proximity Assay: 100 ul of assay volume: 60 ul of cell/beads mix (premixed for at least 30 minutes), 20 ul of I-125-MIP-1α, 20 ul of assay buffer for total binding value, or 20 ul of 0.5 uM MIP-1β for nsb, or 20 ul of test compound. Shake the 96 well plates for 30 minutes on an orbital shaker, then let them settle for 30 minutes before reading with a scintillation counter.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of Formula (I) 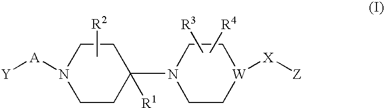
enantiomers, diastereomers or pharmaceutically acceptable salts thereof, wherein Y is a 7 to 10 member bicyclic heterocycle optionally substituted with 1-3 independently selected moieties each of which is R⁵ or R⁶;
A is —CO—, or —SO₂—;
W is N or CH, provided
when W is CH then X is —C(R⁸)₂—, —C(R⁸)(R⁹)—, —C(O)—, —O—, —NH—, —N(C₁-₆ alkyl)-,
—C(R⁸)(OR¹⁰)—, —C(R⁸)(CH₂—C₁-₅alkyl-R¹⁰)—, —C(═CHR¹¹)—,
—C(═NOR¹²)—, —C(R⁸)(O—C₁-₆-alkyl)-, —C(═CH—C₁-₆ alkyl)-,
—C(R⁸)(O—C(O)—C₁-₆ alkyl)-, —C(R⁸)(O—C(O)—O—C₁-₆ alkyl)-,
—C(R⁸)(O—C(O)—NH—C₁-₆ alkyl)-, —C(R⁸)(O—C(O)—N(C₁-₆ alkyl)₂)- —C(R⁸)(NR¹³—C(O)—C₁-₆ alkyl)-, —C(R⁸)(NR¹³—C(O)—O—C₁-₆ alkyl)-,
—C(R⁸)(NR¹³—C(O)—NH—C₁-₆ alkyl)-, —C(R⁸)(NR¹³—C(O)—N—(C₁-6 alkyl)₂)-,
—N(C(O)—C₁-₆ alkyl)-, —C(R⁸)(OH)—, —C(R⁸)(OTMS)-, —CHR⁸—, —CHR¹¹—, —CHR¹⁴—; and
when W is N then X is —C(R⁸ )(R¹⁵)—, or —C(O)—;
Z is R⁷-phenyl, R⁷-pyridyl, R⁷-thiophenyl or R⁷-naphthyl;
R¹ is hydrogen, C₁-₆ alkyl or C₂-₆ alkenyl;
R², R³, R⁴, and R⁸ are each independently hydrogen, C₂-₆ alkenyl, CF₃ or C₁₋₆ alkyl;
R⁵ and R⁶ are independently selected from halogen, C₁₋₆ alkyl, CF₃, nitro, cyano, NR¹³R¹¹, hydroxy, aryl, ester, carboxy, —CO₂R¹¹, OC₁₋₆ alkyl;
R⁷ is 1 to 3 independently selected moieties each of which is hydrogen, halogen, nitro, —NR¹³R¹¹, —CF₃, CF₃O—, —CN, CF₃SO₂—, R¹⁹-phenyl,—NHCOCF₃, C₁-₆ alkyl, —CO₂C₁-₆ alkoxy, 5-membered heteroaryl, CH₃SO₂— or 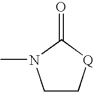
wherein Q is , —O—, —NH— or —N(CH₃)—;
R⁹ is R⁷-phenyl, R⁷-heteroaryl, R⁷-naphthyl, C₃-₁₀ cycloalkyl, C₃-₁₀ cycloalkyl —C₁-₆ alkyl or C₁-₆ alkoxy-C₁-₆ alkyl;
R¹⁰ is R¹⁷-phenyl, pyridyl, pyrimidyl, pyrazinyl or thiazolyl;
R¹¹ is H or C₁-₆ alkyl.
R¹² is hydrogen, —C₁-₆ alkyl, —C₁-₆ alkyl substituted by C₃-₇ cycloalkyl, —C₁-₆ alkyl, fluoro-C₁-₆ alkyl, cyclopropylmethyl-, —CH₂CH₂OH, —CH₂CH₂—O—C₁-₆ alkyl, —CH₂C(O)—O—C₁-₆ alkyl, —CH₂C(O)NH₂, —CH₂C(O)—NHC₁-₆ alkyl, —CH₂CH₂ C₁-₆ alkyl, —CH₂C(O)—C₁-₆ alkyl or —CH₂C(O)—N(C₁-₆ alkyl)₂;
R¹³ is hydrogen or C₁-₆ alkyl;
R¹⁴ is —OH, —CF₃, or O-pyridinyl;
R¹⁵ is hydrogen, C₁-₆ alkyl, C₁-C₆ alkoxy-C₁-₆ alkyl, C₃-₁₀ cycloalkyl, C₃-₁₀ cycloalkyl-C₁-₆ alkyl, R¹⁶-phenyl, R¹⁶-phenyl-C₁-₆ alkyl, R¹⁶-naphthyl, R¹⁶-naphthyl-C₁-₆ alkyl, R¹⁶-heteroaryl or R¹⁶-heteroaryl-C₁-₆ alkyl;
R¹⁶ is 1 to 3 independently selected moieties each of which is hydrogen, halogen, C₁-₆ alkyl, C₁-₆-alkoxy, —CF₃, CF₃O—, CH₃C(O)—, —CN, CH₃SO₂—, CF₃SO₂—, R¹⁸-phenyl, R¹⁸-benzyl, CH₃C(═NOCH₃)—, CH₃C(═NOCH₂CH₃)—, —NH₂, —NHCOCF₃, —NHCONH—(C₁-₆ alkyl), —NHCO(C₁-₆ alkyl), —NHSO₂(C₁-₆ alkyl),5-membered heteroaryl, 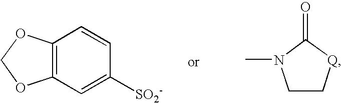
or wherein Q is, —NH— or —N(CH₃);
R¹⁷ is C₁-₆ alkyl, —NH₂ or R¹⁹-phenyl-;
R¹⁸ is 1 to 3 independently selected moieties each of which is hydrogen, C₁-₆-alkyl, —CF₃, —CO₂H, —CO₂C₁-₆ alkoxy, —CN, C₁-₆ alkoxy or halogen;
R¹⁹ is 1 to 3 independently selected moieties each of which is hydrogen, C₁₋₆ alkyl, —CF₃, —CO₂R¹¹, —CN, C₁-₆ alkoxy or halogen.

2. A compound of claim 1 wherein Y is selected from

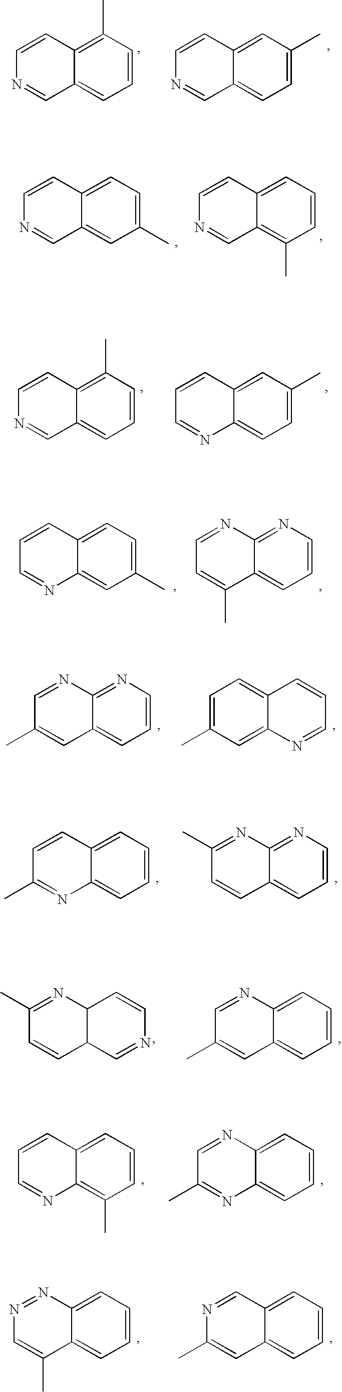

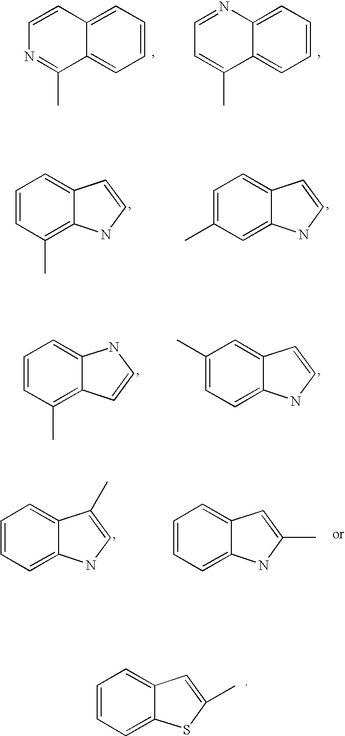

3. A compound of claim 1 wherein Z is bromophenyl, trifluoromethylphenyl, or fluorophenyl.

4. A compound of claim 2 wherein Z is bromophenyl, trifluoromethylphenyl, or fluorophenyl.

5. A compound of claim 1 wherein X is
—C(═NHOEthyl)-
—CH(Opyridinyl)-
—CH(methyl)-
—C(═CH₂)— or
—CH(OH)—.

6. A compound of claim 2 wherein X is
—C(═NHOEthyl)-
—CH(Opyridinyl)-
—CH(methyl)-
—C(═CH₂)— or
—CH(OH)—.

7. A compound of claim 3 wherein X is —C(═NHOEthyl)-
—CH(Opyridinyl)-
—CH(methyl)-
—C(═CH₂)— or
—CH(OH)—.

8. A compound of claim 4 wherein X is
—C(═NHOEthyl)-
—CH(Opyridinyl)-
—CH(methyl)-
—C(═CH₂)— or
—CH(OH)—.

9. A compound of claim 1 wherein R¹ is hydrogen or methyl.

10. A compound of claim 2 wherein R¹ is hydrogen or methyl.

11. A compound of claim 3 wherein R¹ is hydrogen or methyl.

12. A compound of claim 4 wherein R¹ is hydrogen or methyl.

13. A compound of claim 5 wherein R¹ is hydrogen or methyl.

14. A compound of claim 6 wherein R¹ is hydrogen or methyl.

15. A compound of claim 7 wherein R¹ is hydrogen or methyl.

16. A compound of claim 8 wherein R¹ is hydrogen or methyl.

17. A compound of claim 1 selected from
4-[[4-[4-(4-bromobenzoyl)-1-piperidinyl]-4-methyl-1-piperidinyl]carbonyl]-7-chloroquinoline;
1-hydroxy-4-[[4-methyl-4-[(3S)-3-methyl-4-[(1R)-1-[4-(trifluoromethyl)phenyl]ethyl]piperazinyl]-1-piperidinyl]carbonyl]quinolinium;
1-[[4-[4-[(4-bromophenyl)(ethoxyimino)methyl]-1-piperidinyl]-4 methyl-1-piperidinyl]carbonyl]isoquinoline;
3-[[4-[4-[(4-bromophenyl)(ethoxyimino)methyl]-1-piperidinyl]-4-methyl-1-piperidinyl]carbonyl]isoquinoline;
3-[[4-[4-[(4-bromophenyl)(ethoxyimino)methyl]-1-piperidinyl]-4-methyl-1-piperidinyl]carbonyl]quinoline;
2-[[4-[4-[(4-bromophenyl)(ethoxyimino)methyl]-1-piperidinyl]-4-methyl-1-piperidinyl]carbonyl]quinoline;
4-[[4-[4-[(4-bromophenyl)(ethoxyimino)methyl]-1-piperidinyl]-4-methyl-1-piperidinyl]carbonyl]-2-methyl-3-quinolinol;
4-[[4-[4-[(4-bromophenyl)(ethoxyimino)methyl]-1-piperidinyl]-4-methyl-1-piperidinyl]carbonyl]-8-methylquinoline;
4-[[4-[4-[(4-bromophenyl)(ethoxyimino)methyl]-1-piperidinyl]-4-methyl-1-piperidinyl]carbonyl]-6-methylquinoline;
2-[[4-[4-[(4-bromophenyl)(ethoxyimino)methyl]-1-piperidinyl]-4-methyl-1-piperidinyl]carbonyl]-4-quinolinol;
2-[[4-[4-[(4-bromophenyl)(ethoxyimino)methyl]-1-piperidinyl]-4-methyl-1-piperidinyl]carbonyl]-4,8-quinolinediol;
2-[[4-[4-[(4-bromophenyl)(ethoxyimino)methyl]-1-piperidinyl]-4-methyl-1-piperidinyl]carbonyl]-4-methoxyquinoline;
4-[[4-[4-[(E)-(4-bromophenyl)(ethoxyimino)methyl]-1-piperidinyl]-4-methyl-1-piperidinyl]carbonyl]-6-methyl-5-quinolinol;
4-[[4-[4-[(E)-(4-bromophenyl)(ethoxyimino)methyl]-1-piperidinyl]-4-methyl-1-piperidinyl]carbonyl]-7-chloro-6-methylquinoline;
3-[[4-[4-[(4-bromophenyl)(ethoxyimino)methyl]-1-piperidinyl]-4-methyl-1-piperidinyl]carbonyl]-6-(trifluoromethyl)-7-quinolinol;
3-[[4-[4-[(Z)-(4-bromophenyl)(ethoxyimino)methyl]-1-piperidinyl]-4-methyl-1-piperidinyl]carbonyl]-6-(trifluoromethyl)-7-quinolinol;
3-[[4-[4-[(4-bromophenyl)(ethoxyimino)methyl]-1-piperidinyl]-4-methyl-1-piperidinyl]carbonyl]-8-(trifluoromethyl)-4-quinolinol;
2-[[4-[4-[(4-bromophenyl)(ethoxyimino)methyl]-1-piperidinyl]-4-methyl-1-piperidinyl]carbonyl]-6-ethyl-4-quinolinol;
3-[[4-[4-[(4-bromophenyl)(ethoxyimino)methyl]-1-piperidinyl]-4-methyl-1-piperidinyl]carbonyl]-7-(trifluoromethyl)-4-quinolinol;
2-[[4-[4-[(4-bromophenyl)(ethoxyimino)methyl]-1-piperidinyl]-4-methyl-1-piperidinyl]carbonyl]-8-methyl-4-quinolinol;
4-[[4-[4-[(4-bromophenyl)(ethoxyimino)methyl]-1-piperidinyl]-4-methyl-1-piperidinyl]carbonyl]-2-phenylquinoline;
6-[[4-[4-[(E)-(4-bromophenyl)(ethoxyimino)methyl]-1-piperidinyl]-4-methyl-1-piperidinyl]carbonyl]quinoline;
2-[[4-[4-[(4-bromophenyl)(ethoxyimino)methyl]-1-piperidinyl]-4-methyl-1-piperidinyl]carbonyl]-7-ethyl-4-quinolinol;
4-[[4-[4-[(Z)-(4-bromophenyl)(ethoxyimino)methyl]-1-piperidinyl]-4-methyl-1-piperidinyl]carbonyl]quinoline;
4-[[4-[4-[(4-bromophenyl)(ethoxyimino)methyl]-1-piperidinyl]-4-methyl-1-piperidinyl]carbonyl]-7-(trifluoromethyl)quinoline;
4-[[4-[4-[(Z)-(4-bromophenyl)(ethoxyimino)methyl]-1-piperidinyl]-4-methyl-1-piperidinyl]carbonyl]-1-hydroxyquinolinium;
7-[[4-[4-[(Z)-(4-bromophenyl)(ethoxyimino)methyl]-1-piperidinyl]-4-methyl-1-piperidinyl]carbonyl]quinoline;
8-[[4-[4-[(Z)-(4-bromophenyl)(ethoxyimino)methyl]-1-piperidinyl]-4-methyl-1-piperidinyl]carbonyl]quinoline;
4-[[4-[4-[(Z)-(4-bromophenyl)(ethoxyimino)methyl]-1-piperidinyl]-4-methyl-1-piperidinyl]carbonyl]-7-chloroquinoline;
4-[[4-[4-[(Z)-(4-bromophenyl)(ethoxyimino)methyl]-1-piperidinyl]-4-methyl-1-piperidinyl]carbonyl]-7-methylquinoline;
4-[[4-[4-[(E)-(4-bromophenyl)(ethoxyimino)methyl]-1-piperidinyl]-4-methyl-1-piperidinyl]carbonyl]-7-chloro-1-hydroxyquinolinium;
8-[[4-[4-[(Z)-(4-bromophenyl)(ethoxyimino)methyl]-1-piperidinyl]-4-methyl-1-piperidinyl]carbonyl]-4-chloroquinoline;
7-[[4-[4-[(Z)-(4-bromophenyl)(ethoxyimino)methyl]-1-piperidinyl]-4-methyl-1-piperidinyl]carbonyl]-4-chloroquinoline;
4-[[4-[4-[(Z)-(4-bromophenyl)(ethoxyimino)methyl]-1-piperidinyl]-4-methyl-1-piperidinyl]carbonyl]-2-methylquinoline;
5-[[4-[4-[(Z)-(4-bromophenyl)(ethoxyimino)methyl]-1-piperidinyl]-4-methyl-1-piperidinyl]carbonyl]-1-hydroxyquinolinium;
4-[[4-[4-[(E)-(4-bromophenyl)(ethoxyimino)methyl]-1-piperidinyl]-4-methyl-1-piperidinyl]carbonyl]-7-methoxyquinoline;
5-[[4-[4-[(E)-(4-bromophenyl)(ethoxyimino)methyl]-1-piperidinyl]-4-methyl-1-piperidinyl]carbonyl]quinoline;
2-[[4-[[4-[4-[(Z)-(4-bromophenyl)(ethoxyimino)methyl]-1-piperidinyl]-4-methyl-1-piperidinyl]carbonyl]-7-quinolinyl]oxy]ethanol
4-[[4-[4-[(Z)-(4-bromophenyl)(ethoxyimino)methyl]-1-piperidinyl]-4-methyl-1-piperidinyl]carbonyl]-3-methylquinoline;
8-[[4-[4-[(Z)-(4-bromophenyl)(ethoxyimino)methyl]-1-piperidinyl]-4-methyl-1-piperidinyl]sulfonyl]quinoline;
4-[[4-[4-[(4-bromophenyl)(ethoxyimino)methyl]-1-piperidinyl]-4-methyl-1-piperidinyl]carbonyl]cinnoline;
2-[[4-[4-[(4-bromophenyl)(ethoxyimino)methyl]-1-piperidinyl]-4-methyl-1-piperidinyl]carbonyl]quinoxaline;
2-[[4-[4-[(4-bromophenyl)(ethoxyimino)methyl]-1-piperidinyl]-4-methyl-1-piperidinyl]carbonyl]-3-quinoxalinol;
2-[[4-[4-[(4-bromophenyl)(ethoxyimino)methyl]-1-piperidinyl]-4-methyl-1-piperidinyl]carbonyl]-1,6-naphthyridine;
2-[[4-[4-[(4-bromophenyl)(ethoxyimino)methyl]-1-piperidinyl]-4-methyl-1-piperidinyl]carbonyl]-1,8-naphthyridine;
3-[[4-[4-[(E)-(4-bromophenyl)(ethoxyimino)methyl]-1-piperidinyl]-4-methyl-1-piperidinyl]carbonyl]-2-methyl-1,8-naphthyridine;
3-[[4-[4-[(4-bromophenyl)(ethoxyimino)methyl]-1-piperidinyl]-4-methyl-1-piperidinyl]carbonyl]-2-(trifluoromethyl)-1,8-naphthyridine;
3-[[4-[4-[(Z)-(4-bromophenyl)(ethoxyimino)methyl]-1-piperidinyl]-4-methyl-1-piperidinyl]carbonyl]-2-methyl-1,6-naphthyridine;
2-[[4-[4-[(4-bromophenyl)(ethoxyimino)methyl]-1-piperidinyl]-4-methyl-1-piperidinyl]carbonyl]-1H-indole;
3-[[4-[4-[(4-bromophenyl)(ethoxyimino)methyl]-1-piperidinyl]-4-methyl-1-piperidinyl]carbonyl]-1-methyl-1H-indole;
3-[[4-[4-[(4-bromophenyl)(ethoxyimino)methyl]-1-piperidinyl]-4-methyl-1-piperidinyl]carbonyl]-1H-indole;
5-[[4-[4-[(4-bromophenyl)(ethoxyimino)methyl]-1-piperidinyl]-4-methyl-1-piperidinyl]carbonyl]-1H-indole;
5-[[4-[4-[(Z)-(4-bromophenyl)(ethoxyimino)methyl]-1-piperidinyl]-4-methyl-1-piperidinyl]carbonyl]-1-methyl-1H-indole;
5-[[4-[4-[(Z)-(4-bromophenyl)(ethoxyimino)methyl]-1-piperidinyl]-4-methyl-1-piperidinyl]carbonyl]-1-ethyl-1H-indole;
2-[[4-[4-[(E)-(4-bromophenyl)(ethoxyimino)methyl]-1-piperidinyl]-4-methyl-1-piperidinyl]carbonyl]-1-methyl-1H-indole;
2-[[4-[4-[(4-bromophenyl)(ethoxyimino)methyl]-1-piperidinyl]-4-methyl-1-piperidinyl]carbonyl]-1-ethyl-1H-indole;
3-[[4-[4-[(Z)-(4-bromophenyl)(ethoxyimino)methyl]-1-piperidinyl]-4-methyl-1-piperidinyl]carbonyl]-1-ethyl-1H-indole;
4-[[4-[4-[(Z)-(4-bromophenyl)(ethoxyimino)methyl]-1-piperidinyl]-4-methyl-1-piperidinyl]carbonyl]-1-methyl-1H-indole;
6-[[4-[4-[(Z)-(4-bromophenyl)(ethoxyimino)methyl]-1-piperidinyl]-4-methyl-1-piperidinyl]carbonyl]-1-methyl-1H-indole;
4-[[4-[4-[(Z)-(4-bromophenyl)(ethoxyimino)methyl]-1-piperidinyl]-4-methyl-1-piperidinyl]carbonyl]-1-ethyl-1H-indole;
6-[[4-[4-[(Z)-(4-bromophenyl)(ethoxyimino)methyl]-1-piperidinyl]-4-methyl-1-piperidinyl]carbonyl]-1-ethyl-1H-indole;
6-[[4-[4-[(Z)-(4-bromophenyl)(ethoxyimino)methyl]-1-piperidinyl]-4-methyl-1-piperidinyl]carbonyl]-1H-indole;
4-[[4-[4-[(Z)-(4-bromophenyl)(ethoxyimino)methyl]-1-piperidinyl]-4-methyl-1-piperidinyl]carbonyl]-1-methyl-1H-indole;
1-[1-(benzo[b]thien-3-ylcarbonyl)-4-methyl-4-piperidinyl]-4-[(4-bromophenyl)(ethoxyimino)methyl]piperidine;
4-[[4-[4-[(4-bromophenyl)hydroxy-methyl]-4-(4-methyl-4-piperidinyl)-piperidinyl-quinoline;
4-[[4-[4-[(4-bromophenyl)(2-pyridinyloxy)methyl]-1-piperidinyl]-4-methyl-1-piperidinyl]carbonyl]-7-chloroquinoline;
4-[[4-[4-[(4-bromophenyl)(2-pyridinyloxy)methyl]-1-piperidinyl]-4-methyl-1-piperidinyl]carbonyl]quinoline;
4-[[4-[4-[(4-bromophenyl)(2-pyridinyloxy)methyl]-1-piperidinyl]-4-methyl-1-piperidinyl]carbonyl]-1-hydroxyquinolinium;
5-[[4-[4-[(4-bromophenyl)(2-pyridinyloxy)methyl]-1-piperidinyl]-4-methyl-1-piperidinyl]carbonyl]quinoline;
5-[[4-[4-[(4-bromophenyl)(2-pyridinyloxy)methyl]-1-piperidinyl]-4-methyl-1-piperidinyl]carbonyl]-1-hydroxyquinolinium;
4-[[4-[4-[1-(4-bromophenyl)-2,2,2-trifluoro-1-[(trimethylsilyl)oxy]ethyl]-1-piperidinyl]-4-methyl-1-piperidinyl]carbonyl]-7-chloroquinoline;
4-[[4-[4-[1-(4-bromophenyl)-(2,2,2-trifluoro-1-hydroxy)ethyl]-1-piperidinyl]-4-methyl-1-piperidinyl]carbonyl]-7-chloroquinoline;
4-[[4-[4-[1-(4-bromophenyl)ethenyl]-1-piperidinyl]-4-methyl-1-piperidinyl]carbonyl]quinoline; and
1-methyl-4-[[4-methyl-4-[(3S)-3-methyl-4-[(1R)-1-[4-(trifluoromethyl)phenyl]ethyl]piperazinyl]-1-piperidinyl]carbonyl]-1H-indole.

18. A method of treating an inflammatory or immunoregulatory disorder comprising the administration to a patient in need thereof an effective amount of at least one compound of claim 1.

19. A method of claim 18 wherein the inflammatory or immunoregulatory disorder is selected from multiple sclerosis, arthritis, or psoriasis.

20. A method of treating a disorder selected from optic neuritis, uveitis, stroke, endometriosis, dermatitis, inflammatory bowel disease, Crohn's disease, demyelinating disorders, HIV, AIDS dementia complex, transplant rejection, diabetes, alzheimer's disease, cancer and Grave's disease comprising the administration to a patient in need thereof an effective amount of at least one compound of claim 1.

21. A pharmaceutical composition comprising at least one compound of claim 1 together with a pharmaceutically acceptable vehicle or carrier therefor.
